(19) United States
(12) Patent Application Publication
(10) Pub. No.: US 2002/0103145 A1
(43) Pub. Date: Aug. 1, 2002

(54) IMMUNIZATION OF INFANTS

(76) Inventors: ADRIAN BOT, SAN DIEGO, CA (US); CONSTANTIN BONA, NEW YORK, NY (US)

Correspondence Address: BAKER & BOTTS, 30 ROCKEFELLER PLAZA, NEW YORK, NY, 10112

(21) Appl. No.: 09/308,511

(22) PCT Filed: Nov. 21, 1997
(86) PCT No.: PCT/US97/21687

(51) Int. Cl. A61K 48/00; C12N 15/86; C12P 21/02

(52) U.S. Cl. 514/44; 435/375; 435/69.1; 435/456

(57) ABSTRACT

The present invention relates to methods and compositions which may be used to immunize infant mammals against a target antigen, wherein an immunogenically effective amount of a nucleic acid encoding a relevant epitope of a desired target antigen is administered to the infant. It is based, at least in part, on the discovery that such genetic immunization of infant mammals could give rise to effective cellular and humoral immune responses against target antigens.

The invention contained herein was funded, at least in part, by NIH-NIAID grant No. GCO #87-009 MI, so that the United States Government holds certain rights herein.

### 1. INTRODUCTION

The present invention relates to methods and compositions which may be used to immunize infant mammals against one or more target antigens, wherein an immunogenically effective amount of nucleic acid encoding one or more relevant epitopes of one or more desired target antigens is administered to the infant.

### 2. BACKGROUND OF THE INVENTION

A properly operating immune system enables an organism to maintain a healthy status quo by distinguishing between antigens associated with the organism itself, which are allowed to persist, and antigens associated with disease, which are disposed of. Decades ago, Burnet proposed that the immune system's ability to distinguish between “self” and “non-self” antigens results from the elimination of self-reactive lymphocytes in the developing organism (Burnet, 1959, The Clonal Selection Theory of Acquired Immunity, Vanderbilt Univ Press, Nashville, Tenn.). The phenomenon wherein an organism loses the ability to produce an immune response toward an antigen is referred to as “tolerance”.

Over the years, a number of observations consistent with the clonal selection theory of tolerance have been documented. For example, genetically non-identical twin cattle, which share a placenta and are exposed to each other's blood cells in utero, fail to reject the allogeneic cells of their sibling as adults (Owen, 1945, Science 102:400). As another example, adult rodents that had been injected, at birth, with hemopoietic cells from a genetically distinct donor rodent strain were able to accept tissue transplants from that donor strain (Billingham et al., 1953, Nature 172:603; Billingham, 1956, Proc. R. Soc. London Ser. B. 239:44). However, in the early 1980's it was shown that the injection of minute amounts of antigen (namely an immunoglobulin expressing A48 regulatory idiotype) induced the expansion of helper T cells (Rubinstein et al., 1982, J. Exp. Med. 156:506-521).

The concept of tolerization is associated with the traditional belief that neonates are themselves incapable of mounting an effective immune response. It has been generally believed that neonates rely on maternal antibodies (passively transferred via the placenta) for immunity, until the neonate begins to synthesize its own IgG antibodies (at about 3-4 months after birth, in humans; Benjamini and Leskowitz, 1988, “Immunology, A Short Course”, Alan R. Liss, Inc., New York, p. 65).

More recently, several groups have reported findings that dispute the hypothesis that exposure to an antigen in early life disarms the ability of the immune system to react to that antigen.

Forsthuber et al. (1996, Science 271:1728-1730; “Forsthuber”) suggest that the impaired lymph node response of so-called “tolerized” mice was an artifact caused by a technical inability to assess immune function. They reported that neonatal mice, injected with hen egg lysozyme (HEL) in incomplete Freund's adjuvant (“IFA”) according to a protocol considered to induce tolerance in adults as well as neonates, displayed an impaired response in the lymph nodes consistent with tolerization. However, the spleen cells of these mice reportedly proliferated vigorously in response to HEL, a response previously unmeasurable due to technical limitations. The authors propose that neonatal injection did not tolerize, but rather induced functional memory cells that were detectable in spleen but not lymph nodes.

Sarzotti et al. (1996, Science 271:1726; “Sarzotti”) report that inoculation of newborn mice with a high dose of Cas-Br-M murine leukemia virus (“Cas”) does not result in immunological unresponsiveness, but rather leads to a nonproductive type 2 response which is likely to have a negative effect on the induction of mature effector cells. According to Sarzotti, clonal deletion of relevant CTL was not observed in mice infected at birth with a low dose of Cas.

Finally, Ridge et al. (1996, Science 271:1723-1726; “Ridge”) proposes that previous reports of tolerance induction may have been associated with a relative paucity of antigen presenting cells. Ridge observed the induction of CTL reactivity in neonatal mice injected with antigen expressed on dendritic cells (which are so-called professional antigen presenting cells).

The use of nucleic acids as vaccines was known prior to the present invention (see, for example, International Application Publication No. WO 94/21797, by Merck & Co. and Vical, Inc., and International Application Publication No. WO 90/11092). It was not known, however, that such vaccines could be used to induce an immune response in infant mammals.

### 3. SUMMARY OF THE INVENTION

The present invention relates to methods and compositions which may be used to immunize infant mammals against a target antigen, wherein an immunogenically effective amount of nucleic acid encoding one or more relevant epitopes of one or more desired target antigens is administered to the infant. It is based, at least in part, on the discovery that such genetic immunization of infant mammals could give rise to effective cellular (including the induction of cytotoxic T lymphocytes) and humoral immune responses against target antigen. This ability to confer immunity to infants is surprising in the context of the conventional view, that exposure of an infant to an antigen induces tolerance rather than activation of the immune system. In addition, the ability of the present invention to induce a cellular immune response in infants is in contrast to the generally held concept that infants rely on maternal antibodies (rather than cellular elements) for immunity.

Moreover, the present invention may reduce the need for subsequent boost administrations (as are generally required for protein and killed pathogen vaccines), and may prevent side-effects associated with live attenuated vaccines. For instance, the World Health Organization recommends waiting nine months after birth before immunizing against rubella, measles, and mumps, in order to avoid undesirable side effects associated with vaccination against these diseases. Similarly, the World Health Organization recommends waiting two months after birth before immunizing children against influenza virus. In addition to concern over side effects, there is doubt as to whether an effective immune response may be generated using these conventional vaccines prior to the recommended ages.

In preferred embodiments of the invention, nucleic acids encoding more than one relevant epitope of one or more target antigen are administered to an infant mammal for the purposes of genetic immunization. It has been observed that the administration of several epitopes representing distinct target antigens of a pathogen provide a synergistic immune response to the pathogen. Similarly, the administration of multiple epitopes directed to antigens associated with more than one pathogen may be used to provide an infant subject with a broader spectrum of protection. Such an approach may be used to optimize the immunity induced, and may be a means for inducing an immune response to a variety of childhood pathogens.

### 4.DESCRIPTION OF THE FIGURES

FIGS. 1 A-F. Primary and secondary NP-specific cytotoxicity one month after injection of newborn (D-F) or adult (A-C) mice with DNA encoding influenza nucleo-protein (NPV1). The percentage of specific lysis was determined in a standard 4-hour ⁵¹Cr release assay for CTL (cytotoxic T lymphocytes) obtained from newborn or adult animals immunized with NPV1 or control DNA and boosted (or not) with live PR8 virus one month after completing the immunization. An additional control group was injected with saline and boosted one month later with virus. Spleen cells were harvested 7 days after boosting and the percentage of NP-specific cytotoxicity was determined immediately (i.e., primary cytotoxicity) or after incubation for five days with irradiated spleen cells, NP peptide, and IL-2 (i.e. secondary cytotoxicity) as described in Zaghouani et al., 1992, J. Immunol 148, 3604-3609. CTLs were assayed against P815 cells coated with NP peptide (5 μg ml) or infected with PR8 (not shown) or B Lee virus.

FIGS. 2 A-B Limiting dilution assay to determine the frequency of NP-specific CTL precursors one month after injection of newborn (B) and adult (A) mice with NPV1. Splenocytes harvested 7 days after PR8 boosting from newborn and adult mice vaccinated with NPV1 or control plasmid were incubated in serial dilution (6×10⁴ to 2×10¹ splenocytes/well) for 5 days with x-irradiated, PR8-infected splenocytes from non-immunized BALB/c mice in the presence of IL-2 (6 units/ml). The incubation was carried out in 96-well microtiter plates with 24 wells for each dilution of effector cells. Cytotoxicity was assessed against PR8-infected or non-infected P815 cells. Those wells exhibiting percentage lysis greater than background plus three standard deviations were regarded as positive.

FIG. 3. Detection of DNA in muscle of BALB/c mice infected with NPV1. Muscle tissue was removed from the site of injection in the right gluteal muscle of newborns or tibial muscle of adults one month after completion of the vaccination schedule. DNA recovered from the muscle tissue on the left flank of each animal served as a control. The labeling above each lane indicates the origin of DNA.

FIGS. 4 A-C. Cross-reactive CTLS generated in newborns injected with NPV1. The percentage of specific lysis was determined using a standard ⁵¹Cr release assay. Spleen cells were harvested from (A) PR8 immunized mice; (B) genetically immunized newborns that were immunized one month later with PR8 virus and (C) genetically immunized newborns Spleen cells were cultured for 4 days with irradiated PR8-infected spleen cells, then assayed in the presence of ⁵¹Cr-labeled P815 cells noninfected or infected with PR8. A/HK, A/Japan or B lee virus.

FIGS. 5 A-F. Survival of genetically immunized newborn and adult mice challenged 1 mo. (A-D) or 3 mo. (E and F) after immunization with 1.5×10⁴ TCID₅₀ PR8 virus or 3×10⁴ TCID₅₀ HK virus via aerosol.

FIGS. 6 A-B Kinetics of body-weight loss and recover in immunized adult (A) or newborn (B) mice challenged with 1.5×10⁴ TCID50 PR8 virus one month after completing the immunization

FIGS. 7 A-D. Survival of (A-B) newborn and (C-D) adult mice immunized with pHA plasmid encoding hemagglutinin of WSN influenza virus and challenged with LD₁₀₀ of WSN or PR8 virus, 1 month after immunization.

FIGS. 8A-B. Cytotoxicity of splenocytes from mice immunized as neonates with either (A) UV-inactivated influenza virus or (B) pHA+pNP.

FIGS. 9A-C. Secretion of cytokines by CD4+ T cells from mice immunized with (A) pHA+pNP; (B) UV-attenuated WSN virus; and (C) control.

FIGS. 10A-D. Survival of (A) newborn mice immunized with pHA+pNP, pHA, or pNP, challenged with WSN virus; (B) newborn mice immunized with pHA+pNP, pHA or pNP, challenged with PR8 virus; (C) adult mice immunized with pHA+pNP, pHA or pNP, challenged with WSN virus; (D) adult mice immunized with pHA+pNP, pHA or pNP following lethal challenge with PR8 virus.

FIG. 11. Relationship between number of inoculations and protection conferred.

FIG. 12. Lethality of WSN live virus in neonatal BALB/c mice.

FIG. 13. Survival of neonatal mice immunized with UV-attenuated WSN virus.

FIGS. 14A-B. Proliferation of CD4+ T cells (A) stimulated with NP 147-155 peptide or (B) from neonatal mice immunized with VH-TB, boosted with PR8 virus.

FIGS. 15A-D. Secretion of cytokines by T cells from (A) mice having received an inoculation with live PR8 virus, previously immunized (as adults) with VH-TB; (B) mice having received an inoculation with live PR8 virus, previously immunized (as neonates) with VH-TB. (C) mice having received an inoculation with live PR8 virus (no previous immunization). (D) mice having received an inoculation with live PR8 virus (no previous immunization as neonates)

FIG. 16 Cytotoxicity response of mice immunized as neonates with VH-TB.

### 5. DETAILED DESCRIPTION OF THE INVENTION

For purposes of clarity of description, and not by way of limitation, the detailed description of the invention is divided into the following subsections:

(i) compositions for immunization; and

(ii) methods of immunization.

### 5.1. Compositions for Immunization

The present invention provides for compositions which may be used to immunize infant mammals against one or more target antigens which comprise an effective amount of a nucleic acid encoding one or more relevant epitopes of the target antigen(s) in a pharmaceutically acceptable carrier. Following administration of the compositions, transformed host cells will express the relevant antigens, thereby provoking the desired immune response.

Nucleic acids which may be used herein include deoxyribonucleic acid (“DNA”) as well as ribonucleic acid (“RNA”). It is preferable to use DNA in view of its greater stability to degradation.

The term “target antigen” refers to an antigen toward which it is desirable to induce an immune response. Such an antigen may be comprised in a pathogen, such as a viral, bacterial, protozoan, fungal, yeast, or parasitic antigen, or may be comprised in a cell, such as a cancer cell or a cell of the immune system which mediates an autoimmune disorder. For example, but not by way of limitation, the target antigen may be comprised in an influenza virus, a cytomegalovirus, a herpes virus (including HSV-I and HSV-II), a vaccinia virus, a hepatitis virus (including but not limited to hepatitis A, B, C, or D), a varicella virus, a rotavirus, a papilloma virus, a measles virus, an Epstein Barr virus, a coxsackie virus, a polio virus, an enterovirus, an adenovirus, a retrovirus (including, but not limited to, HIV-1 or HIV-2), a respiratory syncytial virus, a rubella virus, a Streptococcus bacterium (such as Streptococcus pneumoniae), a Staphylococcus bacterium (such as Staphylococcus aureus), a Hemophilus bacterium (such as Hemophilus influenza), a Listeria bacterium (such as Listeria monocytogenes), a Klebsiella bacterium, a Gram-negative bacillus bacterium, an Escherichia bacterium (such as Escherichia coli), a Salmonella bacterium (such as Salmonella typhimurium), a Vibrio bacterium (such as Vibrio cholerae),a Yersinia bacterium (such as Yersinia pestis or Yersinia enterocoliticus), an Enterococcus bacterium, a Neisseria bacterium (such as Neiserria meningitidis), a Corynebacterium bacterium (such as Corynebacterium diphtheriae), a Clostridium bacterium (such as Clostridium tetani), a Mycoplasma (such as Mycoplasma pneumoniae), a Pseudomonas bacterium, (such as Pseudomonas aeruginosa), a Mycobacteria bacterium (such as Mycobacterium tuberculosis), a Candida yeast, an Aspergillus fungus, a Mucor fungus, a toxoplasma, an amoeba, a malarial parasite, a trypanosomal parasite, a leishmanial parasite, a helminth, etc. Specific nonlimiting examples of such target antigens include hemagglutinin, nucleoprotein, M protein, F protein, HBS protein, gp 120 protein of HIV, nef protein of HIV, and listeriolysine. In alternative embodiments, the target antigen may be a tumor antigen, including, but not limited to, carcinoembryonic antigen (“CEA”), melanoma associated antigens, alpha fetoprotein, papilloma virus antigens, Epstein Barr antigens, etc..

The term “relevant epitope”, as used herein, refers to an epitope comprised in the target antigen which is accessible to the immune system. For example, a relevant epitope may be processed after penetration of a microbe into a cell or recognized by antibodies on the surface of the microbe or microbial proteins. Preferably, an immune response directed toward the epitope confers a beneficial effect; for example, where the target antigen is a viral protein, an immune response toward a relevant epitope of the target antigen at least partially neutralizes the infectivity or pathogenicity of the virus. Epitopes may be B-cell or T-cell epitopes.

The term “B cell epitope”, as used herein, refers to a peptide, including a peptide comprised in a larger protein, which is able to bind to an immunoglobulin receptor of a B cell and participates in the induction of antibody production by the B cells.

For example, and not by way of limitation, the hypervariable region 3 loop (“V3 loop”) of the envelope protein of human immunodeficiency virus (“HIV”) type 1 is known to be a B cell epitope. Although the sequence of this epitope varies, the following consensus sequence, corresponding to residues 301-319 of HIV-1 gp120 protein, has been obtained: Arg-Lys-Ser-Ile-His-Ile-Gly-Pro-Gly-Arg-Ala-Phe-Tyr-Thr-Thr-Gly-Glu-Ile-Ile (SEQ ID NO:1)

Other examples of known B cell epitopes which may be used according to the invention include, but are not limited to, epitopes associated with influenza virus strains, such as Trp-Leu-Thr-Lys-Lys-Gly-Asp-Ser-Tyr-Pro (SEQ ID NO:2), which has been shown to be an immunodominant B cell epitope in site B of influenza HA1 hemagglutinin, the epitope Trp-Leu-Thr-Lys-Ser-Gly-Ser-Thr-Tyr-Pro (H3; SEQ ID NO:3), and the epitope Trp-Leu-Thr-Lys-Glu-Gly-Ser-Asp-Tyr-Pro (H2; SEQ ID NO:4) (Li et al., 1992, J. Virol. 66:399-404); an epitope of F protein of measles virus (residues 404-414; Ile-Asn-Gln-Asp-Pro-Asp-Lys-Ile-Leu-Thr-Tyr; SEQ ID NO:5; Parlidos et al., 1992, Eur. J. Immunol. 22:2675-2680); an epitope of hepatitis virus pre-S1 region, from residues 132-145 (Leclerc, 1991, J. Immunol. 147:3545-3552); and an epitope of foot and mouth disease VP1 protein, residues 141-160, Met-Asn-Ser-Ala-Pro-Asn-Leu-Arg-Gly-Asp-Leu-Gln-Lys-Val-Ala-Arg-Thr-Leu-Pro (SEQ ID NO:6; Clarke et al., 1987, Nature 330:381-384).

Still further B cell epitopes which may be used are known or may be identified by methods known in the art, as set forth in Caton et al., 1982, Cell 31:417-427.

In additional embodiments of the invention, peptides which may be used according to the invention may be T cell epitopes. The term “T cell epitope”, as used herein; refers to a peptide, including a peptide comprised in a larger protein, which may be associated with MHC self antigens and recognized by a T cell, thereby functionally activating the T cell.

For example, the present invention provides for Tₕ epitopes, which, in the context of MHC class II self antigens, may be recognized by a helper T cell and thereby promote the facilitation of B cell antibody production via the Tₕ cell.

For example, and not by way of limitation, influenza A hemagglutinin (HA) protein of PR8 strain, bears, at amino acid residues 110-120, a Tₕ epitope having the amino acid sequence Ser-Phe-Glu-Arg-Phe-Glu-Ile-Phe-Pro-Lys-Glu (SEQ ID NO:7).

Other examples of known T cell epitopes include, but are not limited to, two promiscuous epitopes of tetanus toxoid. Asn-Ser-Val-Asp-Asp-Ala-Leu-Ile-Asn-Ser-Thr-Lys-Ile-Tyr-Ser-Tyr-Phe-Pro-Ser-Val (SEQ ID NO:8) and Pro-Glu-Ile-Asn-Gly-Lys-Ala-Ile-His-Leu-Val-Asn-Asn-Glu-Scr-Ser-Glu (SEQ ID NO:9; Ho et al., 1990, Eur. J. Inmmunol. 20:477-483); an epitope of cytochrome c, from residues 88-103, Ala-Asn-Glu-Arg-Ala-Asp-Leu-Ile-Ala-Tyr-Leu-Gln-Ala-Thr-Lys (SEQ ID NO: 10); an epitope of Mycobacteria heatshock protein, residues 350-369, Asp-Gln-Val-His-Phe-Gln-Pro-Leu-Pro-Pro-Ala-Val-Val-Lys-Leu-Ser-Asp-Ala-Leu-Ile (SEQ ID NO: 11; Vordermir et al., Eur. J. Immunol. 24:2061-2067); an epitope of hen egg white lysozyme, residues 48-61, Asp-Gly-Ser-Thr-Asp-Tyr-Gly-Ile-Leu-Gln-Ile-Asn-Ser-Arg (SEQ ID NO: 12; Neilson et al., 1992, Proc. Natl. Acad. Sci. U.S.A. 89:7380-7383); an epitope of Streptococcus A M protein, residues 308-319, Gln-Val-Glu-Lys-Ala-Leu-Glu-Glu-Ala-Asn-Ser-Lys (SEQ ID NO:13; Rossiter et al., 1994, Eur. J. Immunol. 24:1244-1247); and an epitope of Staphylococcus nuclease protein, residues 81-100, Arg-Thr-Asp-Lys-Tyr-Gly-Arg-Gly-Leu-Ala-Tyr-Ile-Tyr-Ala-Asp-Gly-Lys-Met-Val-Asn (SEQ ID NO:14; de Magistris, 1992, Cell 68:1-20). Still further Tₕ epitopes which may be used are known or may be identified by methods known in the art.

As a further example, a relevant epitope may be a T_{CTL} epitope, which, in the context of MHC class I self antigens, may be recognized by a cytotoxic T cell and thereby promote CTL-mediated lysis of cells comprising the target antigen. Nonlimiting examples of such epitopes include epitopes of influenza virus nucleoproteins TYQRTRALVRTGMDP (SEQ ID NO: 15) or IASNENMDAMESSTL (SEQ ID NO: 16) corresponding to amino acid residues 147-161 and 365-379, respectively (Taylor et al., 1989 Immunogenetics 26 267). Townsend et al., 1983, Nature 348:674); LSMV peptide, KAVYNFATM, amino acid residues 33-41 (SEQ ID NO:17; Zinhernagel et al., 1974, Nature 248:701-702); and oval bumin peptide, SIINFEKL, corresponding to amino acid residues 257-264 (SEQ ID NO 18, Cerbone et al., 1983. J. Exp. Med. 163:603-612).

The nucleic acids of the invention encode one or more relevant epitopes, and may optionally further comprise elements that regulate the expression and/or stability and/or immunogenicity of the relevant epitope For example, elements that regulate the expression of the epitope include, but are not limited to, a promoter/enhancer element, a transcriptional initiation site, a polyadenlation site, a transcriptional termination site, a ribosome binding site, a translational start codon, a translational stop codon, a signal peptide, etc. Specific examples include, but are not limited to, a promoter and intron A sequence of the initial early gene of cytomegalovirus (CMV or SV40 virus (“SV40”);

Montgomery et al., 1993, DNA and Cell Biology 12:777-783). With regard to enhanced stability and/or immunogenicity of the relevant epitope, it may be desirable to comprise the epitope in a larger peptide or protein. For example, and not by way of limitation, the relevant epitope may be comprised in an immunoglobulin molecule, for example, as set forth in U.S. patent application Ser. No. 08/363,276, by Bona et al., the contents of which is hereby incorporated in its entirety herein by reference. Alternatively, more than one epitope may be expressed within the same open reading frame.

Nucleic acids encoding the relevant epitope(s) and optionally comprising elements that aid in its expression, stability, and/or immunogenicity may be comprised in a cloning vector such as a plasmid, which may be propagated using standard techniques to produce sufficient quantities of nucleic acid for immunization. The entire vector, which may preferably be a plasmid which is a mammalian expression vector comprising the cloned sequences, may be used to immunize the infant animal. Sequences encoding more than one epitope of one or more target antigens may be comprised in a single vector.

Examples of nucleic acids which may be used according to the invention are set forth in International Application Publication No. WO 94/21797, by Merck & Co. and Vical, Inc., U.S. Pat. Nos. 5,589,466 and 5,580,859 and in International Application Publication No WO 90/11092, by Vical, Inc., the contents of which are hereby incorporated in their entireties herein by reference.

Different species of nucleic acid, encoding more than one epitope of one or more target antigens, may be comprised in the same composition or may be concurrently administered as separate compositions The term “different species”, as used herein. refers to nucleic acids having different primary sequences. For example, a composition of the invention may comprise one species of nucleic acid encoding a first epitope and a second species of nucleic acid encoding a second epitope, with multiple molecules of both species being present The term “effective amount”, as used herein, refers to an amount of nucleic acid encoding at least one relevant epitope of at least one target antigen, which, when introduced into a infant mammal, results in a substantial increase in the immune response of the mammal to the target antigen. Preferably, the cellular and/or humoral immune response to the target antigen is increased, following the application of methods of the invention, by at least four-fold, and preferably by at least between 10-fold and 100-fold (inclusive), above baseline. The immunity elicited by such genetic immunization may develop rapidly after the completion of the immunization (e.g., within 7 days), and may be long lasting (e.g., greater than 9 months). The need for “boosting” in order to achieve an effective immune response may be diminished by the present invention. In preferred embodiments, the effective amount of nucleic acid is introduced by multiple inoculations (see below).

In specific, nonlimiting embodiments of the invention, nucleic acid encoding between 1-500 picomoles of relevant epitope, preferably between 20-100 picomoles of relevant epitope, and more preferably between 40-100 picomoles of relevant epitope per gram weight of the infant mammal may be administered.

Thus, in selected embodiments the compositions of the present invention may comprise strands of nucleic acids encoding more than one relevant epitope. As explained herein, the relevant epitopes may be found in the same target antigen, in different antigens from the same pathogen or in unrelated target antigens from different pathogens. With respect to the latter, opportunistic pathogens may be targeted along with the primary disease causing agent. In addition to the broad target range, the disclosed compositions may comprise various epitope combinations. For example, the compositions of the present invention may comprise nucleic acids encoding mixtures of B cell epitopes, mixtures of T cell epitopes, or combinations of B and T cell epitopes. Regardless of which type of epitopes are selected, it will be appreciated that the relevant epitopes may be encoded on the same nucleic acid molecule (i.e., a plasmid) and may even be expressed within the same open reading frame. Alternatively, relevant epitopes may be encoded by separate, non-covalently bound nucleic acid molecules which may be administered in combination as a vaccine “cocktail”. In particularly preferred embodiments these combination vaccines will comprise one or more species of plasmid, each encoding at least one relevant epitope.

As will be demonstrated by the appendant examples, genetic vaccination of infants using compositions comprising nucleic acid molecules (whether as a single species or as a combination of species) which express more than one relevant epitope may exhibit an unexpected synergistic effect. More particularly, such combination vaccines may prove to be much more efficient at conferring the desired immunity with respect to the selected pathogen(s) than compositions comprising a single nucleic acid species encoding a single relevant epitope. Those skilled in the art will appreciate that such synergism could allow for an effective immunoprophylactic or immunotherapeutic response to be generated with lower dosing and less frequent administration than single-epitope DNA vaccines. Moreover, the use of such multi-epitope DNA vaccine compositions may provide more comprehensive protection as the induced multi-site immunity would tend to be more resistant to natural phenotypic variation within a species or rapid mutation of a target antigen by the selected pathogen. Of course, effective immunity may also be imparted by DNA vaccines encoding a single B or T cell epitope and such compositions are clearly contemplated as being within the scope of the present invention.

In addition to nucleic acids, the compositions of the invention may comprise a pharmaceutically acceptable carrier, such as, for example, but not limited to, physiologic saline or liposomes. In specific, nonlimiting embodiments, the concentration of nucleic acid preferably ranges from 3-100 μg/100 μl. In certain embodiments, it may be desirable to formulate such compositions as suspensions or as liposomal formulations.

### 5.2 Methods of Immunization

The present invention provides for a method for immunizing an infant mammal against one or more target antigen, comprising inoculating the mammal with an effective amount of nucleic acid(s) encoding relevant epitope(s) of the target antigen(s) in a pharmaceutically acceptable carrier

The term “infant”, as used herein, refers to a human or non-human mammal during the period of life following birth wherein the immune system has not yet fully matured. In humans, this period extends from birth to the age of about nine months, inclusive. In mice, this period extends from birth to about four weeks of age. The terms “newborn” and “neonate” refer to a subset of infant mammals, which have essentially just been born. Other characteristics associated with “infants” according to the invention include an immune response which has (i) susceptibility to high zone tolerance (deletion/energy of T cell precursors, increased tendency for apoptosis); (ii) a Th2 biased helper response (phenotypical particularities of neonatal T cells; decreased CD40L expression on neonatal T cells); (iii) reduced magnitude of the cellular response (reduced number of functional T cells; reduced antigen-presenting cell function); and (iv) reduced magnitude and restricted isotype of humoral response (predominance of IgM^{high} IgD^{low} B cells, reduced cooperation between Th and B cells). In specific nonlimiting embodiments of the invention, nucleic acid immunization may be administered to an infant animal wherein maternal antibodies remain present in detectable amounts.

In specific nonlimiting embodiments of the invention, nucleic acid immunization may be administered to an infant mammal wherein maternal antibodies remain present in detectable amounts. In a related embodiment, the pregnant mother may be immunized with a nucleic-acid based vaccine prior to delivery so as to increase the level of maternal antibodies passively transferred to the fetus.

The terms “immunize” or “immunization” or related terms refer herein to conferring the ability to mount a substantial immune response (consisting of antibodies or cellular immunity such as effector CTL) against a target antigen or epitope. These terms do not require that completely protective immunity be created, but rather that a protective immune response be produced which is substantially greater than baseline. For example, a mammal may be considered to be immunized against a target antigen if the cellular an/or humoral immune response to the target antigen occurs following the application of methods of the invention Preferably, immunization results in significant resistance to the disease caused or triggered by pathogens expressing target antigens.

The term “innoculating”, as used herein, refers to introducing a composition comprising at least one species of nucleic acid according to the invention into a infant animal. As mentioned above, the composition may comprise more than one nucleic acid species directed to one or more relevant epitopes found on one or more target antigen. The introduction of the selected composition may be accomplished by any means and route known in the art, including intramuscular, subcutaneous, intravenous, intraperitoneal, intrathecal, oral, nasal, rectal, etc. administration. Preferably, inoculation is performed by intramuscular injection.

The effective amount of nucleic acid is preferably administered in several inoculations (that is to say, the effective amount may be split into several doses for inoculation). The number of inoculations is preferably at least one, and is more preferably three.

The success of the inoculations may be confirmed by collecting a peripheral blood sample from the subject between one and four weeks after immunization and testing for the presence of CTL activity and/or a humoral response directed against the target antigen, using standard immunologic techniques.

In specific, nonlimiting embodiments, the present invention may be used to immunize a human infant as follows. A human infant, at an age ranging from birth to about 9 months, preferably at an age ranging from birth to about 6 months, more preferably at an age ranging from birth to about 1 month, and most preferably at an age ranging from birth to about 1 week, may commence a program of injections whereby the infant may be injected intramuscularly three times at 3-7 day intervals with a composition comprising 1-100 nanomoles of DNA encoding a relevant epitope(s) of target antigen(s), preferably at a DNA concentration of 1-5 mg/100 μl, wherein the target antigen may be a protein from a pathogen, for example respiratory syncytial virus, rotavirus, influenza virus, hepatitis virus, or HIV virus (see above).

Accordingly, the present invention provides for compositions for use in immunizing an infant mammal against one or more target antigens, comprising one or more species of nucleic acid encoding one or more epitopes of said target antigen(s) in an amount effective in inducing a cellular (e.g. CTL) and/or humoral immune response.

It is believed that one of the advantages of the present invention is that mammals immunized by such methods may exhibit a lesser tendency to develop an allergy or other adverse reaction after exposure to target antigens. Further, DNA vaccination of infants may reduce the risk of tolerance induction following other vaccination protocols which require successive administration of relatively high doses of antigen.

In preferred embodiments (see Example 7, infra), the present invention provides for a method for immunizing an infant animal against one or more pathogen comprising inoculating the mammal with an effective amount of nucleic acid(s) encoding more than one relevant epitope of one or more target antigen associated with the pathogen(s) in a pharmaceutically acceptable carrier, such that therapeutically effective amounts of the relevant epitopes are expressed in the infant mammal. Analogous methods may be used to induce immunity to undesirable cells or organisms which are not pathogens.

### 6. EXAMPLE

### Induction of Cellular Immunity against Influenza Virus Nucleoprotein in Newborn Mice by Genetic Vaccination

### 6.1. Materials and Methods

Plasmids. The NPV1 plasmid (obtained from Dr. Peter Palese) was constructed by inserting a cDNA derived from the nucleoprotein gene of A/PR8/34 into the BglII site of a mutated pBR322 vector, namely pCMV-IE-AKi-DHFR (Whong et al., 1987, J. Virol. 61:1796), downstream from a 1.96 kb segment of the enhancer, promoter and intron A sequence of the initial early gene of cytomegalovirus and upstream of a 0.55 kb segment of the β globin polyadenylation signal sequence as described in Ulmer et al., 1993. Science 259:1745 The modified pBR322 vector without the NP sequence (termed the “V1 plasmid”) was employed as a control. PRc/CMV-HA/WSN plasmid (pHA plasmid or WSN-HA plasmid) was constructed by inserting HA of A/WSN/33 (subtype H1N1) strain of influenza virus into the PRc CMV mammalian expression vector and donated by Dr. Peter Palese (Mount Sinai School of Medicine). All plasmids were propagated in Escherichia coli and purified by the alkaline lysis method (Id.).

Viruses. The influenza virus strains A/PR8/34 (H1N1). A/HK/68(H3N2), A/Japan/305/57(H2N2) and B Lee 40 were grown in the allantoic cavity of embryonated hen eggs as described in Kilbourne, 1976, J. Infect. Dis. 134:384-394. The A/HK/68 virus adapted to mice was provided by Dr. Margaret Liu (Merck Research Laboratories). The influenza virus strain A/WSN/33 was grown in MDBK cells and purified from supernatants.

Immunization. One month old adult mice were vaccinated with 30 μg of NPV1, pHA or control plasmid dissolved in 100 μl of physiologic saline by injection into the anterior tibial muscle of the shaved right leg using a disposable 28 gauge insulin syringe that was permitted to penetrate to a depth of 2 mm; three injections with 30 μg DNA were carried out at three week intervals. Newborn mice were immunized with 30 μg of plasmid dissolved in 50 μl of physiologic saline by similar injection into the right gluteal muscle of Days 1, 3 and 6 after birth of life. Some newborn mice were injected intraperitoneally (“IP”) on Day 1 after birth with PR8 or B Lee live virus (5 μg in 0.1 ml saline). One month after completion of the vaccination schedule, some mice were boosted with live virus in saline at a dose of 1×10³ TCID₅₀ injected ip.

Infection. Mice were challenged via the aerosol route with 1.5×10⁴ TCID₅₀ of A/PR8/34 (LD100) or 3.2×10⁵ TCID₅₀ of A/HK/68 (LD₁₀₀ virus) or 3×10⁷ TCID₅₀ of A/WSN/33 (LD₁₀₀). Exposure was carried out for 30 minutes in an aerosol chamber to which a nebulizer (Ace Glass, Inc.) was attached via a vacuum/pressure system pump operated at a rate of 35 L/min and a pressure of 15 lb/in². Mice were observed once daily post-infection and their survival was recorded.

Viral lung titers. Processing of lung tissue was carried out with at least three mice from each treatment group as described in as described in Isobe et al., 1994, Viral Immunol. 7:25-30, and viral titers in lung homogenates were determined using an MDCK cell-chicken RBC hemagglutination assay.

Cytotoxic assay. A primary cytotoxicity assay was carried out by incubating effector cells with 5×10^{1 51}Cr-labeled target cells at different effector-to-target ratios in 96-well V-bottom plates P815 target cells were infected with PR8 virus for 1 hour before labeling with ⁵¹Cr or incubated during the assay with 5-10 μg/ml of NP₁₄₇₋₁₅₅. After incubation for 4 hours at 37° C. in 5% CO₂, the supernatant was harvested and radioactivity released was determined using a gamma counter. A secondary cytotoxicity assessment was carried out after co-culturing equal numbers of lymphocytes from test animals and x-irradiated, virus-infected or NP₁₄₇₋₁₅₅-coated lymphocytes from non-immunized BALB c mice for five days in RPMI supplemented with fetal calf serum (“FCS”) 10% and 50 μM 2-mercaptoethanol; the secondary CTL assay itself was conducted using the ⁵¹Cr release assay described above, and the results were expressed as the percentage of specific lysis determined in triplicate for each effector:target ratio employed, as follows:

100(actual−spontaneous release)−(maximum−spontaneous release−background release)±SD

Limiting dilution analysis of CTL precursors. The number of antigen-specific CTL precursors in the spleens of immunized mice were assessed by incubating single-cell suspensions of splenic responder cells in six steps of two-fold dilutions with 2.5×10⁵ X-irradiated, PR8-infected syngeneic splenocytes. After five days in complete RPMI medium, individual microtiter cultures were assayed using ⁵¹Cr release from P815 cells infected with influenza virus, uninfected P815 cells were used as a control. Those wells exhibiting ⁵¹Cr release greater than background plus three standard deviations were regarded as positive. The percentage of cultures in one dilution step regarded as negative for specific cytotoxicity were plotted logarithmically against the number of responder cells/well, and the frequency of CTL precursors was determined by linear regression analysis using the following formula:

1n(negative-well index)−(number of responder cells/well)=1(number of responder cells well at 0.37 negative well index).

The number of precursor cell is represented as 1/frequency for purposes of comparison.

Plasmid detection by PCR. Injected and control muscle tissue was removed one month after completion of the vaccination schedule, immediately frozen in ethanol-dry ice, and stored at −80° C. Frozen tissue was homogenized in lysis buffer and DNA was extracted as described in Montgomery, 1993, DNA and Cell Biol. 12:777-783 and Ulmer et al., 1993. Science 254 1745 A forty-cycle PCR reaction was carried out with NP-specific primers located at the following nucleotide positions: 1120 (minus strand; 5′-[CATTGTCTAGAATTTGAACTCCTCTAGTGG]-3′; SEQ ID NO:19) as well as 468 (positive strand; 5′-[AATTTGAATGATGCAAC]-3′; SEQ ID NO:20). A PCR product with a specific signal of 682 bp was visualized using ethidium bromide stained agarose gels.

Hemagglutination inhibition assay. Sera from immunized mice were treated with receptor destroying enzyme (RDE/neuraminidase) for 1 hour at 37° C. in a waterbath. Two-fold serial dilutions of RDE-treated sera were incubated with 0.5% human erythrocyte saline suspension in the presence of hemagglutinating titers of influenza virus. The experiment was carried out in triplicate wells. After 45 minutes incubation in a 96-well round bottom RIA plates (Falcon) at room temperature, the results were read and expressed as log₂ of the last inhibitory dilution. Negative controls (blank sera) and positive controls (HA specific monoclonal antibodies) were included in the experiment.

Cytokine measurement by ELISA. T cells were incubated, for four days, with antigen and irradiated accessory cells, and then 100 microliters of supernatant were harvested from each microculture. The concentrations of IFN gamma and IL-4 were measured using ELISA test kits (Cytoscreen, from Biosource Int. and Interest from Genzyme, respectively). Standards with known concentrations were included in the assay. The optical densities were assessed at 450 nm absorbance after blanking the ELISA read on the null concentration wells.

### 6.2 Results

Priming of CTL precursors via neonatal DNA vaccination. The optimal schedule for DNA vaccination in the experiments described was developed in pilot studies. Newborn mice were immunized with 30 μg of NPV1 or control plasmid on Days 1, 3 and 6 after birth, adult animals were vaccinated with the same amount of DNA immunogen on Days 0, 21 and 42 of the study. One month after the completion of this standard series of vaccinations, certain test animals were boosted with live PR8 virus.

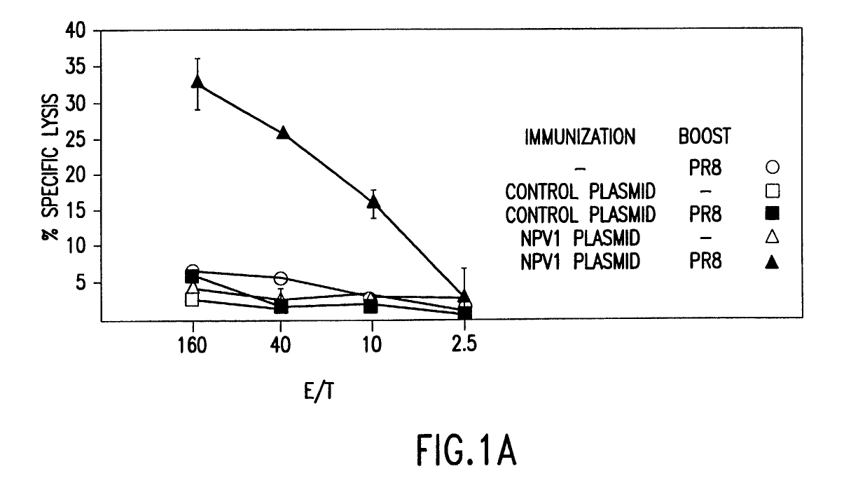

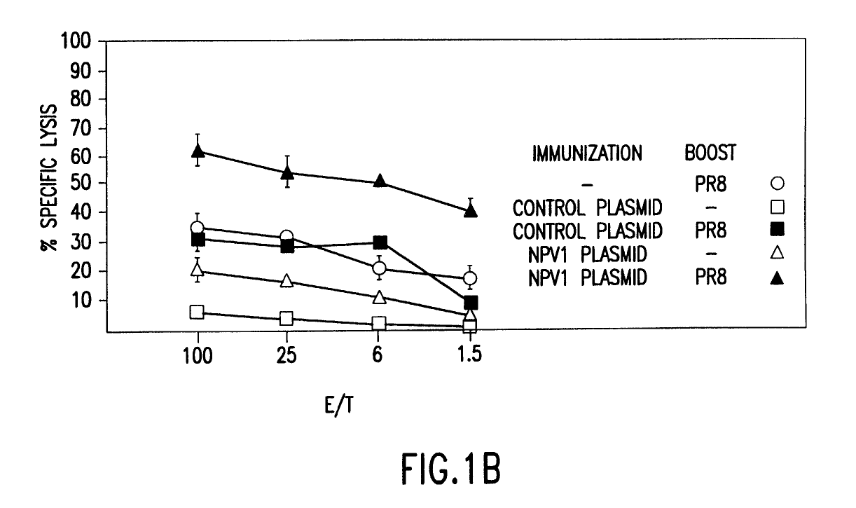

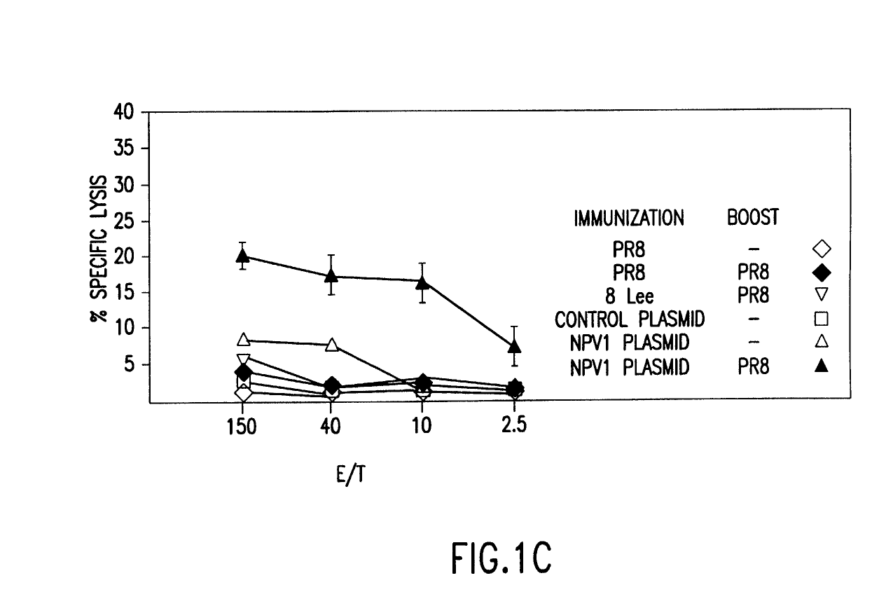

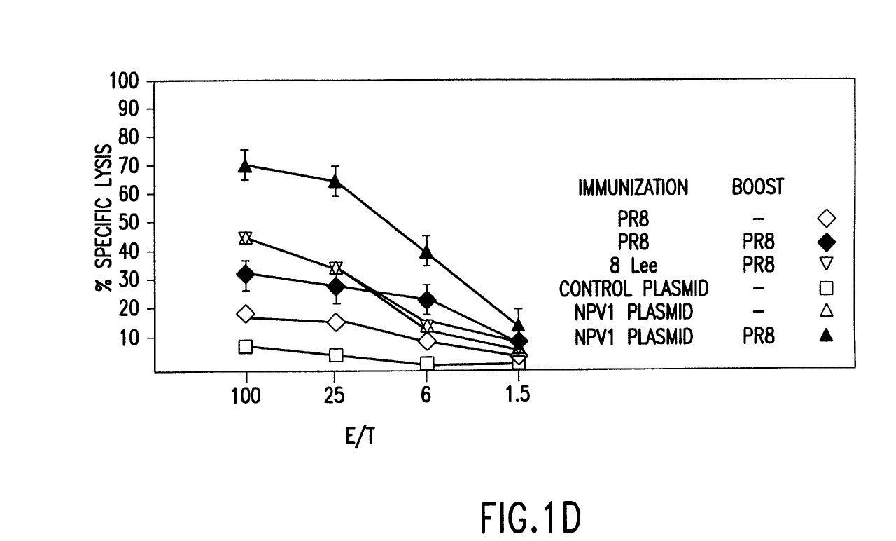

The lymphocytes, directly isolated from newborn and adult mice vaccinated with NPV1 and boosted with PR8 virus, lysed target cells coated in vitro with NP₁₄₇₋₁₅₅, which is recognized by CTL in association with K^{d} MHC-molecules of Class I (FIGS. 1 A-F). No primary cytotoxicity was observed in vitro with lymphocytes from newborns immunized on Day 1 with PR8 virus and boosted one month later with PR8 virus. As expected, significant cytotoxicity was observed after in vitro expansion of splenocytes from mice immunized with NP-V1 plasmid or PR8 virus only. No significant cytotoxicity was observed in the case of mice immunized with control virus or B/Lee virus. These data clearly indicate that vaccination with NPV1 with or without subsequent boosting with native virus induced an expansion of NP-specific CTL precursors in both newborn animals and adults; however, both primary cytotoxicity and immunologically significant secondary cytotoxicity were observed only in animals fully immunized with NPV1 and boosted with virus.

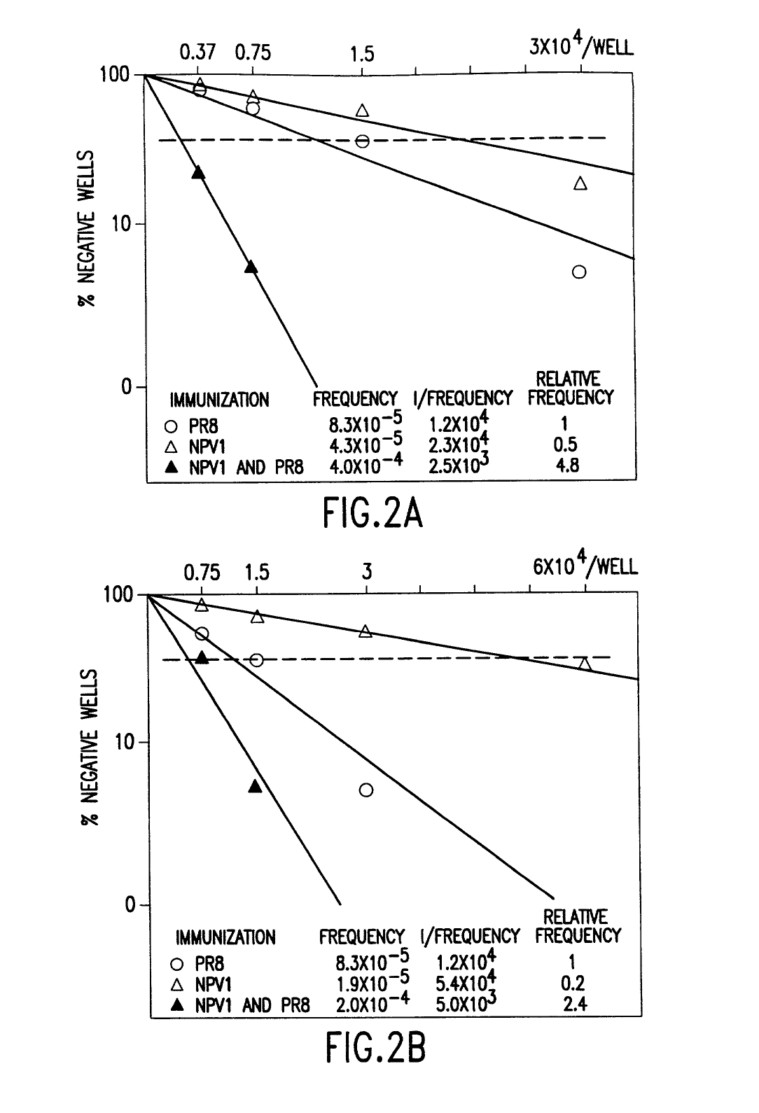

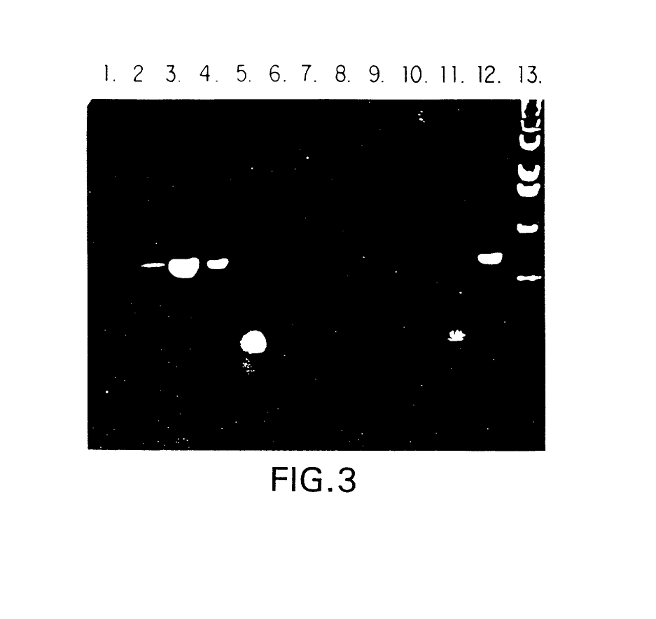

Frequency of NP-specific CTL precursors. An immunologically significant increase in the frequency of NP-specific CTL precursors was observed in animals immunized with NPV1 and boosted with PR8 virus, accounting for the presence of primary cytotoxicity in this particular group (FIGS. 2 A-B). The increased frequency of specific precursors is presumably due to sustained biosynthesis of NP antigen, which primed and expanded this population of NP-specific lymphocytes. Plasmid was detected by qualitative PCR one month after completion of the immunization series in gluteal muscle, the site of injection of NPV1 in newborns, and in tibial muscle, the site of injection of NPV1 in adult animals (FIG. 3).

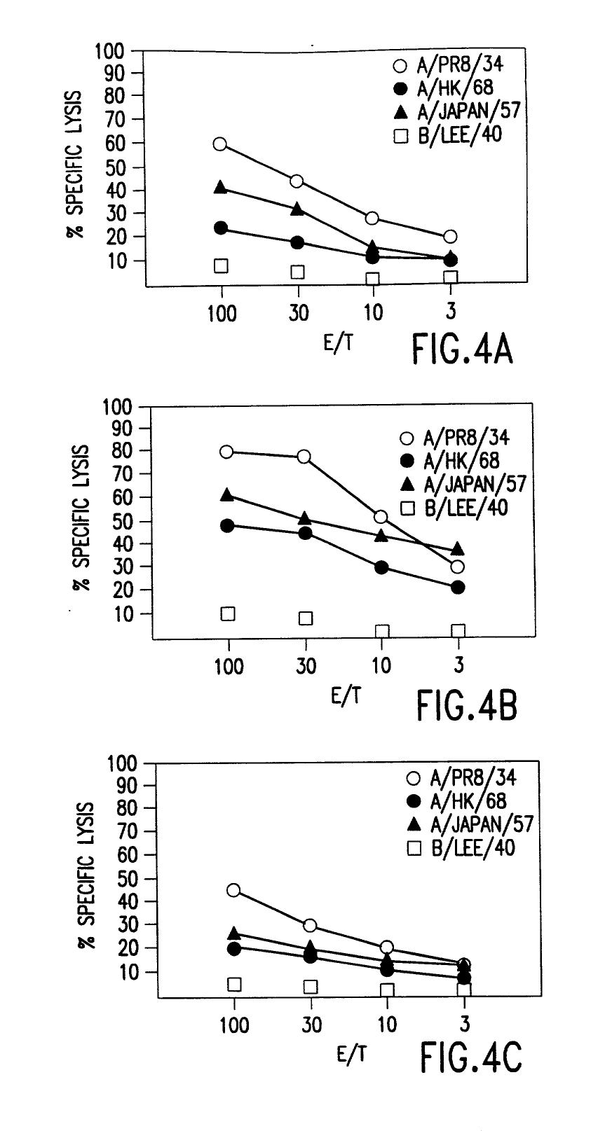

Induction of cross-reactive CTLs via DNA immunization. The induction of cross-reactive CTLs against NP-subtypes in adult animals immunized with type A influenza virus is well-characterized and understood to be related to the limited genetic variation of NP compared to hemagglutinin (HA) and neuraminidase (NA), which are viral surface proteins In a similar manner, CTLs derived from newborn mice immunized with NPV1 and boosted with PR8 virus exhibited increased lysis of P815 cells infected with a variety of influenza strains, including PR8(H₁N₁). A/HK/68(H₃N₂) and A/Japan(H₂N₂) viruses, but not the Type B virus B/Lee, after in vitro stimulation with PR8 virus infected cells or NP₁₄₇₋₁₅₅ peptide (FIGS. 4 A-C).

Effect of DNA immunization on pulmonary virus titer. The increased activity of CTLs in those animals vaccinated with NPV1 is correlated with decreased viral titers in lung tissue measured after aerosol challenge with one LD₁₀₀ of PR8 or HK viruses. Although no difference in viral titers .as observed in mice immunized with NPV1 or control plasmid three days after PR8 challenge, a statistically significant reduction was observed in both newborn (p<0.05) and adult mice (p<0.025; Table I) seven days after challenge. No virus was detected in the lungs of mice that survived challenge for more than 16 days. It is important to note that decreased viral titers in lung tissue were observed in mice challenged with PR8 virus one or three months after completing the immunization (p<0.05).

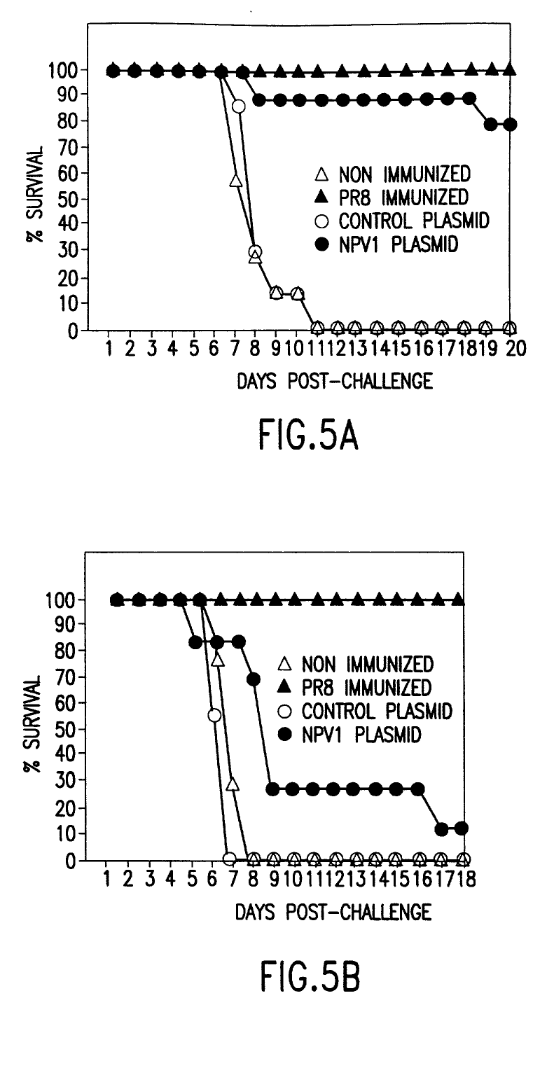

Effect of DNA immunization on clinical course of infection and survival. Genetic immunization of adult mice with NPV1 induced protective immunity in 80% of animals challenged with PR8 virus one month after the last immunization (p<0.01; FIGS. 5A-D) and in 57 percent of adult animals challenged three months after the last immunization (p<0.05; FIG. 5E). An increased survival after challenge was observed in three month old mice immunized with NPV1 as newborns, indicating that during the three month period a more vigorous expansion of CTL precursors was elicited after genetic immunization (p<0.02, FIG. 5B). Only 10% of adult animals challenged with HK virus survived (FIGS. 5A-D), findings that differ from those previously reported (Ulmer et al., 1993. Science 259: 1745-1749) even though the DNA immunogen and Hk strain used in challenge were identical. The relative decrease in survival we observed could be explained by the intranasal route of challenge used previously (Id.), which is less likely to provide productive infection of the lower as well as upper respiratory tract compared to the aerosol challenge employed in these studies. Despite their immuno-responsiveness, one-month old mice immunized with NPV1 as newborns exhibited reduced survival after challenge with PR8 and no survival after challenge with HK virus compared to immunized adults and three month old mice infected with NPV1 as newborns, which exhibited significant survival after challenge with LD100 of PR8 virus.

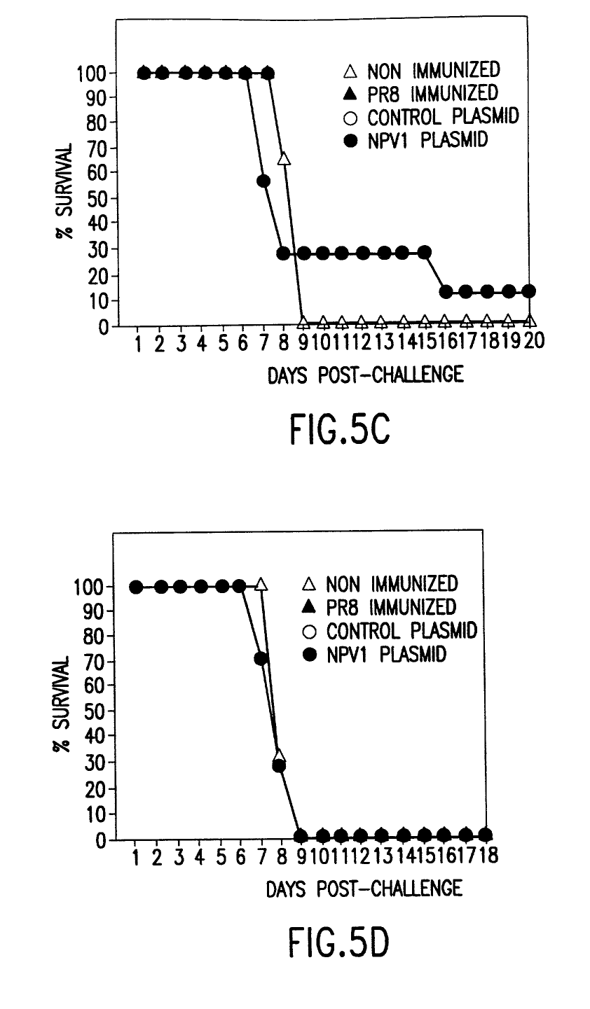

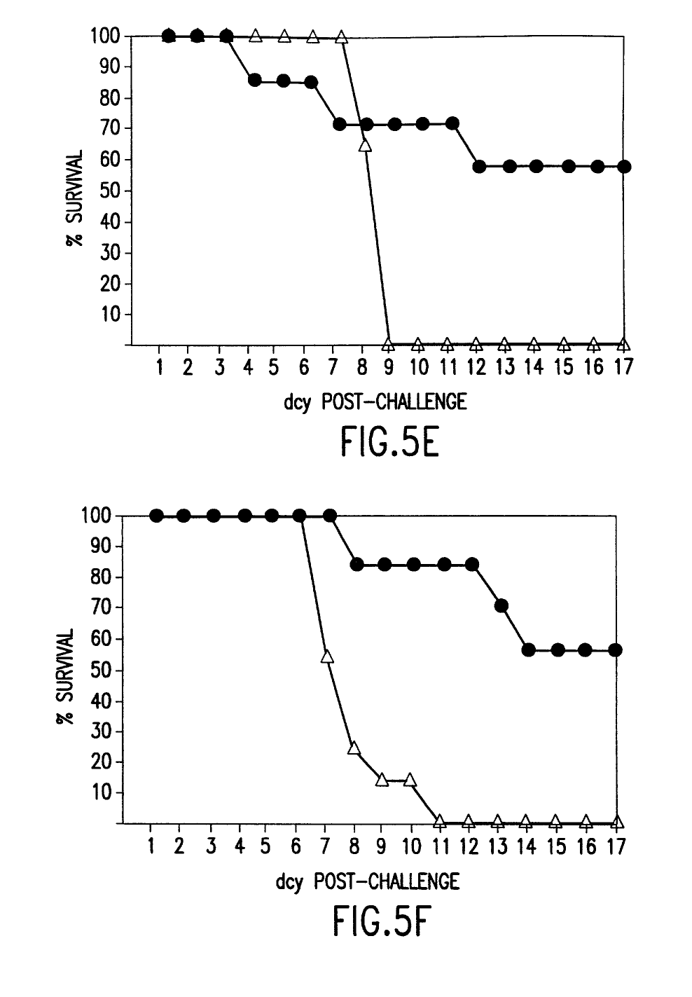

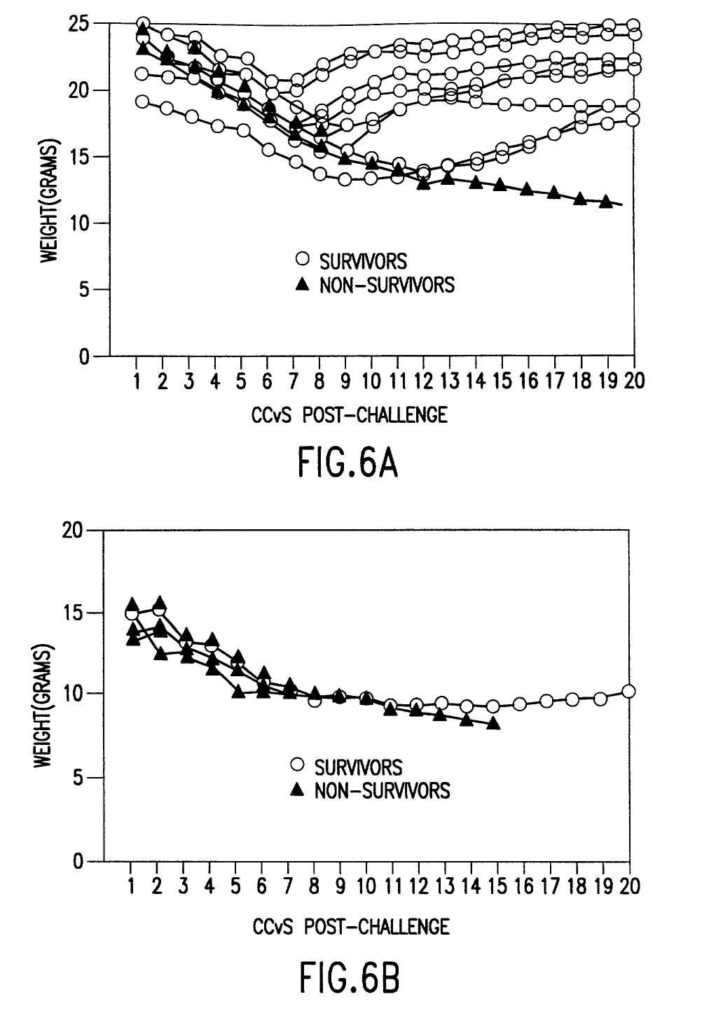

The pneumonia that occurs after influenza infection is accompanied by weight loss in these animals. Adult mice treated with control plasmid and challenged with a lethal dose of PR8 gradually lost weight until they expired (Days 7-9), while the surviving animals immunized with NPV1 recovered their prechallenge body weight by Day 10 after significant initial weight loss post-challenge (Day 2-7; FIGS. 6A-B). Similar results were obtained with one-month old mice which had been immunized after birth as newborns with NPV1 (FIGS. 6 A-B) or with three month old mice.

Effect of DNA immunization with a plasmid which encodes HA of influenza virus (pHA plasmid). Immunization of newborn mice with pHA according to the same protocol as NPV1 was followed by specific antibody production as early as 1 month after birth which persisted at least three months after birth (Table 2). These antibodies displayed hemagglutination inhibiting properties, like antibodies obtained by live-virus or plasmid immunization of adult mice. In consequence, immunization of neonates with pHA elicited protective, virus-specific antibodies.

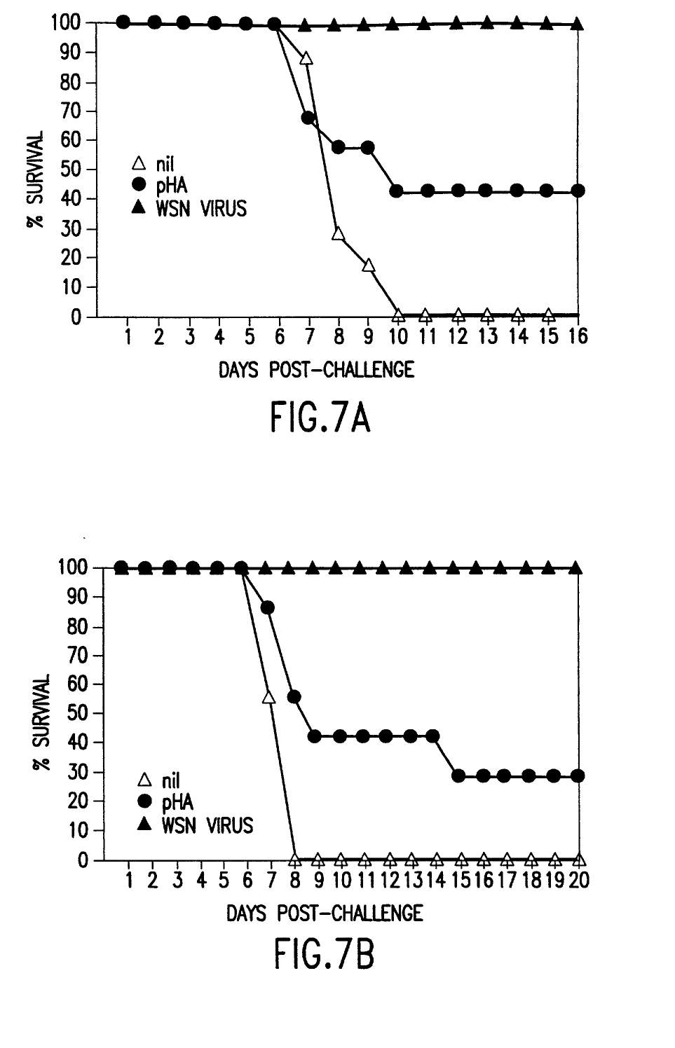

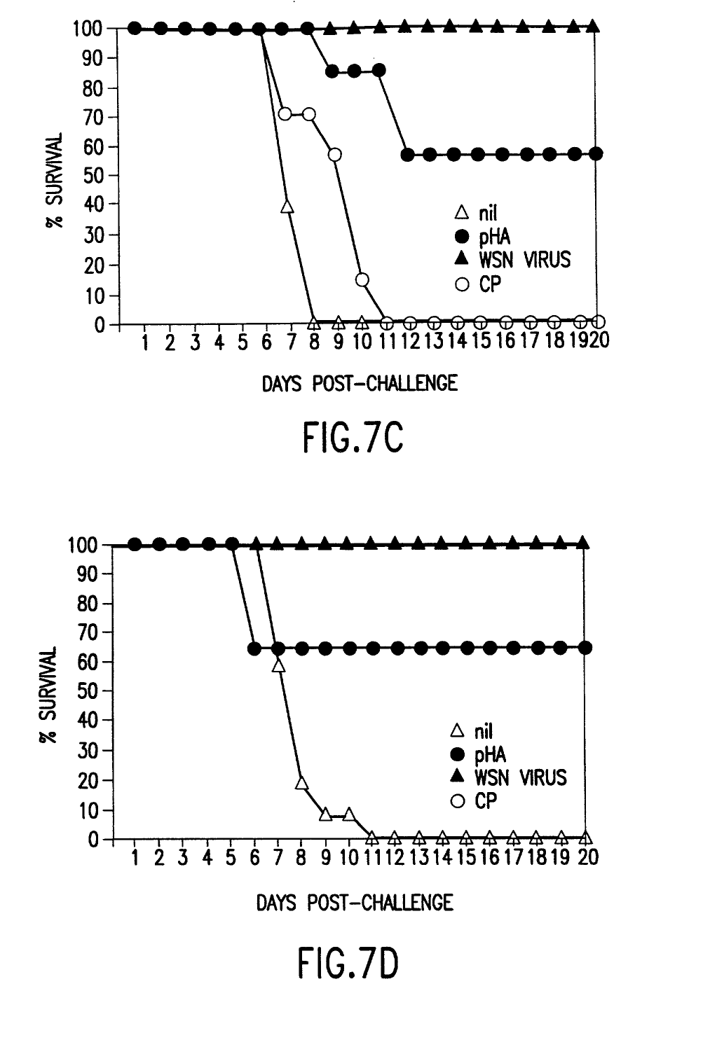

Immunization of mice with pHA primed T helper cells which were then able to secrete cytokines upon in vitro restimulation with virus (Table 3). Whereas pHA injection of adult mice elicited predominantly TH1 type cells, innoculation of neonates with the same plasmid lead to the development of a mixed Th1/Th2 response. DNA immunization of neonates as well as adult mice with pHA conferred significant protection to lethal challenge (LD₁₀₀ ) with WSN or PR8 virus as early as one month after immunization (FIGS. 7A-D).

### 6.3. Discussion

Numerous studies have indicated that the genetic immunization of adult mice, chickens, ferrets and monkeys with cDNAs containing NP or HA sequences of various strains of type A influenza virus can induce protective cellular and humoral immunity (Ulmer et al., 1993, Science 258 1745-1749. Montgomery et al., 1993, DNA and Cell Biol. 11:777-783, Fyneu et al. 1993, Proc Natl. Acad. Sci. U.S.A. 90:11478-11482; Justevicz et al., 1995. J Virol 19 7712-7717, Donnely et al., 1995, Nature Med. 1:583-587). The results presented herein are the first evidence that such immunization has a comparable effect in newborn animals, and that cellular immunity is generated consequent to a strong priming effect characterized by a significant increase in the frequency of antigen-specific CTL precursors. The survival after challenge, the reduction in viral lung titers and recovery of prechallenge body weight compared to controls in animals that were vaccinated with NPV1 or pHA is indicative of effective secondary immune responses.

Previous studies in adult mice have indicated that immunization with homologous virus affords 100% protection to lethal challenge, while only 50-60% protection occurs in normal mice infused with NP-specific T cell clones (Taylor et al., 1986, Immunology 58:417-420) or in PR8-immunized B cell deficient (J_{H}D−/−) animals (Bot et al., 1996, J. Virol., 70:5668-5672), indicating that effective protection requires both humoral and cellular responsiveness, the former presumably mitigating the spread of virus and the extent of pulmonary lesions. The absence of a protective antibody response in the studies carried out with NPV1 plasmid as well as slow expansion of CTL precursors during the first month of life may explain the relatively poor survival of one month old mice that were immunized with NPV1 plasmid as newborns. The increased survival of three month old mice immunized as newborns with NPV1 plasmid suggests that the expansion of CTL precursors continues after neonatal immunization, enabling the mice to develop a stronger cellular response when they become adults.

Further data indicates that the plasmid expressing the HA gene of WSN virus, injected after birth, elicits both humoral and cellular responses mirrored in an increased survival. For example, neonatal immunization with pHA triggered an antibody response associated with a helper response which conferred significant protection upon later challenge with influenza virus

TABLE 1
EFFECT OF IMMUNIZATION WITH NPV1 PLASMID
ON PULMONARY VIRUS TITER MEASURED AFTER
CHALLENGE WITH LETHAL DOSES OF PR8 OR HK VIRUS

| | | challenge with | | | | | |
|---|---|---|---|---|---|---|---|
| | | 1.5 × 10⁴ | | | challenge with | | |
| age of | | TCID₅₀ PR8 virus | | | TCID₅₀HK virus | | |
| animals | immunization | 3d | 7d | 16d | 3d | 7d | 16d |
| adult | nil | 4.6 ± | 3 8 ± | +³ | 6.4 ± | 5.7 ± | + |
| | | 0.5 | 0.1 | | 0.7 | 0.3 | |
| | PR8 virus | 0 | 0 | ND | 5.7 ± | 0 | ND |
| | | | | | 0.3 | | |
| | control plasmid | 4 8 ± | 3.7 ± | + | 6.8 ± | 5.7 | + |
| | | 0 1 | 0.5 | | 0.1 | | |
| | NPV1-1 month¹ | 4 0 ± | 0.9 ± | 0⁴ | 5.8 ± | 0.6 ± | 0 |
| | | 0 3 | 1.5 | | 0.1 | 1.1 | |
| | NPV1-3 months² | 4 8 ± | 0.2 ± | 0 | 6.9 ± | 4.6 ± | 0 |
| | | 0.1 | 0.2 | | 0.7 | 0.8 | |
| newborn | control plasmid | 5 9 ± | 4 6 ± | + | ND | ND | ND |
| | | 0 | 0.2 | | | | |
| | NPV1-1 month | 4 5 ± | 1 2 ± | 0 | 6.6 ± | 5.1 ± | + |
| | | 1.2 | 2.1 | | 0.3 | 0 6 | |
| | NPV1-3 months | 4 1 ± | 0 9 ± | 0 | ND | ND | ND |
| | | 0 5 | 1.2 | | | | |

Mice were sacrificed 1 month after the last immunization. Data are expressed as log₁₀ of viral titer in TCID units
ND-not done
¹mice challenged 1 month after completing the immunization
²mice challenged 3 months after completing the immunization
³no survivors at day 16 after challenge
⁴pulmonary virus titer in mice which survived more than 16 days

TABLE 2
III TITER OF BALB/C MICE IMMUNIZED WITH WSN VIRUS OR PLASMIDS

| Mice | | | Prebleeding | | | | | No. of | | | Titer 7 days after | |
|---|---|---|---|---|---|---|---|---|---|---|---|---|
| immunized | Immunization | No. of | Titer | | Time of | Titer against: | | respondents | | | boost against: | |
| as: | with | mice: | WSN | PR8 | bleeding: | WSN | PR8 | WSN | PR8 | Boost | WSN | PR8 |
| Adults | WSN | 5 | 0^{a} | 0 | 1 mo. | 8.2 ± 1.1^{b} | 1.2 ± 0.8 | 5/5 | 5/5 | WSN | 8.2 ± 1 3 | 2.2 ± 1.6 |
| | CP | 3 | 0 | 0 | 1 mo. | 0 | 1.0 ± 0 7 | 0/3 | 1/3 | — | 0 | 0 |
| | CP | 3 | 0 | 0 | 1 mo. | 0 | 0 | 0/3 | 0/3 | WSN | 7.3 ± 5.3 | 1.3 ± 2 3 |
| | pHA | 16 | 0 | 0 | 1 mo. | 5.5 ± 3.4 | 0 | 12/16 | 0/16 | WSN | 8.3 ± 1.5 | 1 0 ± 1.9 |
| | pHA | 8 | 0 | 0 | 3 mo. | 8.7 ± 3.8 | 0 | 5/8 | 0/8 | WSN | 8.3 ± 1.5 | 2 0 ± 2 0 |
| | pHA | 9 | 0 | 0 | 6 mo. | 10 ± 0 | 0 | 2/9 | 0/9 | WSN | 8 3 ± 0 6 | 1 3 ± 0 6 |
| | pHA | 3 | 0 | 0 | 9 mo. | 0 | 0 | 0/3 | 0/3 | WSN | 5 6 ± 0 6 | 5 0 ± 1 7 |
| Newborns | CP | 5 | ND | ND | 1 mo. | 0 | 0 | 0 5 | 0 5 | WSN | 7 0 ± 0 8 | 0 |
| | pHA | 19 | ND | ND | 1 mo. | 5 2 ± 2 7 | 0 | 12 19 | 0 19 | WSN | 9 4 ± 0 9 | 2 0 ± 1 6 |
| | pHA | 4 | ND | ND | 3 mo | 3 3 ± 1 5 | 0 | 1 4 | 0 4 | WSN | 8 8 ± 2 9 | 3 2 ± 2 5 |

^{a}0 = <1:40
^{b}log₂ dilution
ND—not done

TABLE 3
Lymphokine production by T cells from mice immunized with pHA
plasmid or WSN virus:

| Group | | | Adult mice | | Newborn mice | |
|---|---|---|---|---|---|---|
| Immunization | Boost | Lymphokines | ml* | WSN* | ml | WSN |
| ml | — | IFNγ | 0 | 0 | ND | ND |
| | — | IL-4 | 0 | 0 | ND | ND |
| CP | — | IFNγ | 0 | 11 ± 5** | 14 ± 5 | 22 ± 3 |
| | — | IL-4 | 0 | 0 | 0 | 0 |
| | WSN | IFNγ | 24 ± 1 | 158 ± 4 | 89 ± 28 | 261 ± 26 |
| | WSN | IL-4 | 236 ± 11 | 79 ± 19 | 198 ± 5 | 141 ± 39 |
| pHA | — | IFNγ | 9 ± 1 | 60 ± 2 | 0 | 29 ± 18 |
| | — | IL-4 | 0 | 0 | 2 + 2 | 6 ± 3 |
| | WSN | IFNγ | 19 ± 3 | 284 ± 10 | 38 ± 8 | 179 ± 50 |
| | WSN | IL-4 | 54 ± 3 | 31 ± 4 | 138 ± 4 | 257 ± 24 |
| WSN | — | IFNγ | 52 ± 2 | 214 ± 11 | 103 ± 30 | 51 ± 8 |
| | — | IL-4 | 48 ± 3 | 181 ± 3 | 132 ± 6 | 248 ± 20 |
| | WSN | IFNγ | 10 ± 1 | 127 ± 3 | 9 ± 5 | 61 ± 12 |
| | WSN | IL-4 | 218 ± 4 | 235 ± 12 | 228 ± 8 | 594 ± 5 |

*1.5 × 10 nylon wool non-adherent splenocytes were incubated for four days with 1.5 × 10⁵ irradiated BALD c splenocytes with or without 10 μg ml UV-innactivated WSN virus, in presence of IU ml exogenous IL-2
**concentration of cytokines in supematant was determined by ELISA and expressed as pg ml Values below background ± 3 × SD were considered 0.

### 7. EXAMPLE

### Neonatal Immunization with a Mixture of Plasmids Expressing HA and NP Influenza Virus Antigens

The experiments described above showed that neonatal immunization of BALB/c mice with plasmids expressing NP or HA of Influenza virus is followed by priming of B, Th and CTL rather than tolerance. However, protection in terms of survival against lethal challenge with homologous or heterologous strains was not complete. Further, in the case of NP expressing plasmid, the protective immunity required a longer time to develop following neonatal inoculation, as compared to adult immunization.

In order to improve the protection conferred by plasmid vaccines, we coinjected pHA together with pNP in newborn and adult mice as a so-called “cocktail”. Each of these plasmids, which together encode the entire HA and NP proteins, produce antigens comprising Th, B and CTL epitopes. We challenged the mice at the age of 5 weeks with LD₁₀₀ of WSN virus or the drift variant, PR8 virus.

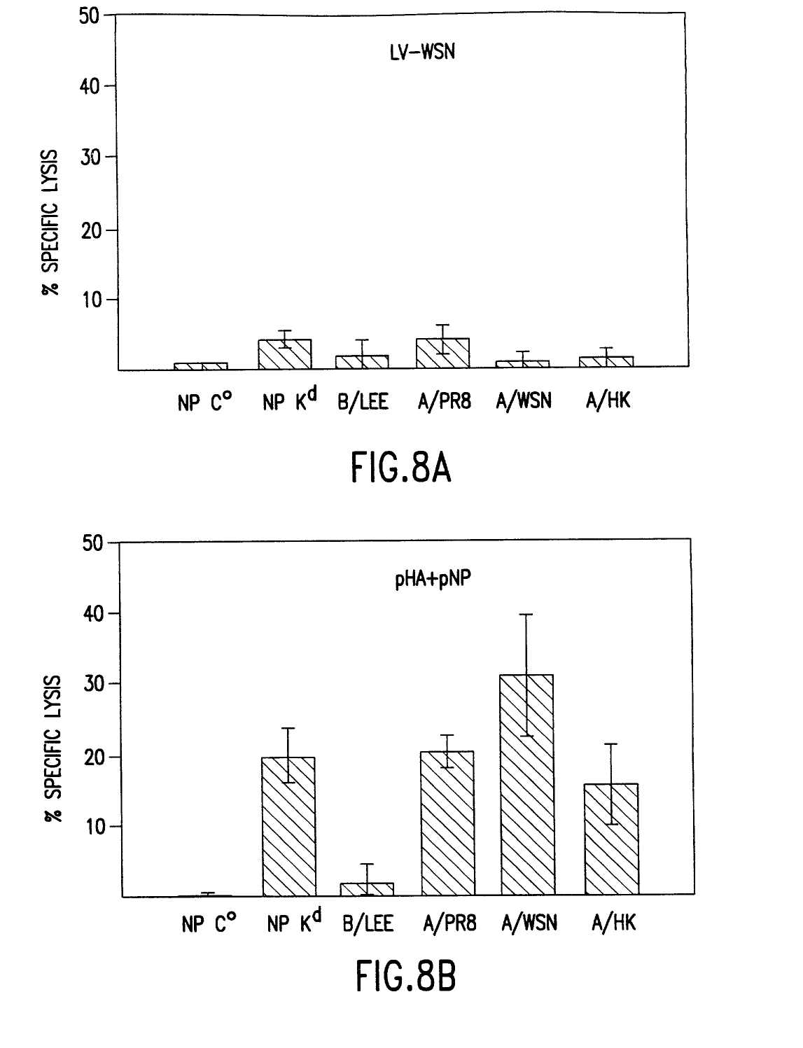

CTL and Th induced by neonatal inoculation of pHA+pNP or UV-attenuated WSN virus. The cytotoxic immunity and the cytokine profile of T cells from mice immunized as neonates with pHA+pNP or from mice immunized with UV-attenuated WSN virus was studied. FIG. 8 depicts the CTL response of mice immunized as newborns (infants) with either (A) UV-attenuated WSN virus or (B) a combination of pHA and pNP plasmids. Splenocytes pooled from three mice in each group were in vitro stimulated with PR8-virus infected APC and tested against P815 cells coated with NP peptides or infected with various influenza viruses at E/T ratio of 10:1. The results are expressed as means of percent specific lysis plus or minus the standard deviation of triplicates. Splenocytes from mice immunized as neonates with UV inactivated virus did not exhibit cytotoxicity against a panel of type A Influenza viruses or against the dominant NP K^{d} epitope, following in vitro stimulation with PR8 infected APC (FIG. 8A). In contrast, neonatal immunization with pHA+pNP primed a significant cytotoxic response against H1N1 strains like PR8 and WSN, against HK that is an H3N2 strain and against the dominant CTL epitope, NP 147-155 (FIG. 8B). No response was detected against a type B virus or a peptide that binds to D^{b} instead of K^{d} class I molecules. further detailed, we could not test the responsiveness of newborn mice to live WSN virus, because of its lethality. In sharp contrast, live virus immunization of adult mice with WSN virus induced high titers of HI antibodies against the homologous virus. Immunization of adult mice with UV-attenuated virus or pHA+pNP induced smaller HI titers against the homologous virus (Table 4). In all cases, the HI titers against the drift variant namely PR8 virus, were not significant.

TABLE 4
HEMAGGLUTINATION-INHIBITION TITERS OF SERA
FROM MICE IMMUNIZED AS NEONATES WITH pHA + pNP

| Age of | Immunized | Number of | HI titer of antibodies against^{a} | |
|---|---|---|---|---|
| immunization | with | Mice | WSN | PR8 |
| Adult | Nil | 2 | 0^{b} | 0 |
| | UV-WSN | 3 | 4 7 ± 0 6 | 0 |
| | hve WSN | 3 | 7 0 ± 1.0 | 0 |
| | pHA-pNP | 3 | 3 3 ± 1.1 | 0 |
| Neonatal | Nil | 2 | 0 | 0 |
| | UV-WSN | 3 | 0 | 0 |
| | pHA-pNP | 5 | 2.2 ± 0.8 | 0 |

^{a}Results were expressed as means of log individual HI titers ± SE.
^{b}Titers less than 1 40 were considered 0
^{c}Results shown for the five responder mice out of the eight mice tested.

Thus, neonatal immunization with pHA+pNP induced suboptimal but significant titers of HI antibodies in a subset of animals. In contrast, neonatal inoculation with UV-attenuated WSN virus was not effective in inducing detectable titers of protective antibodies.

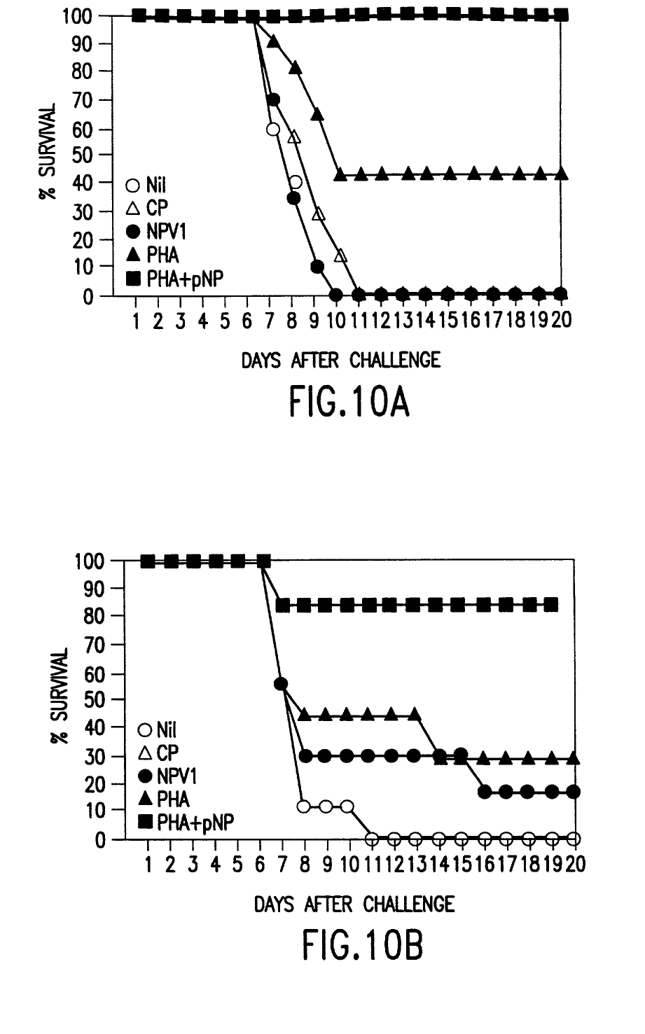

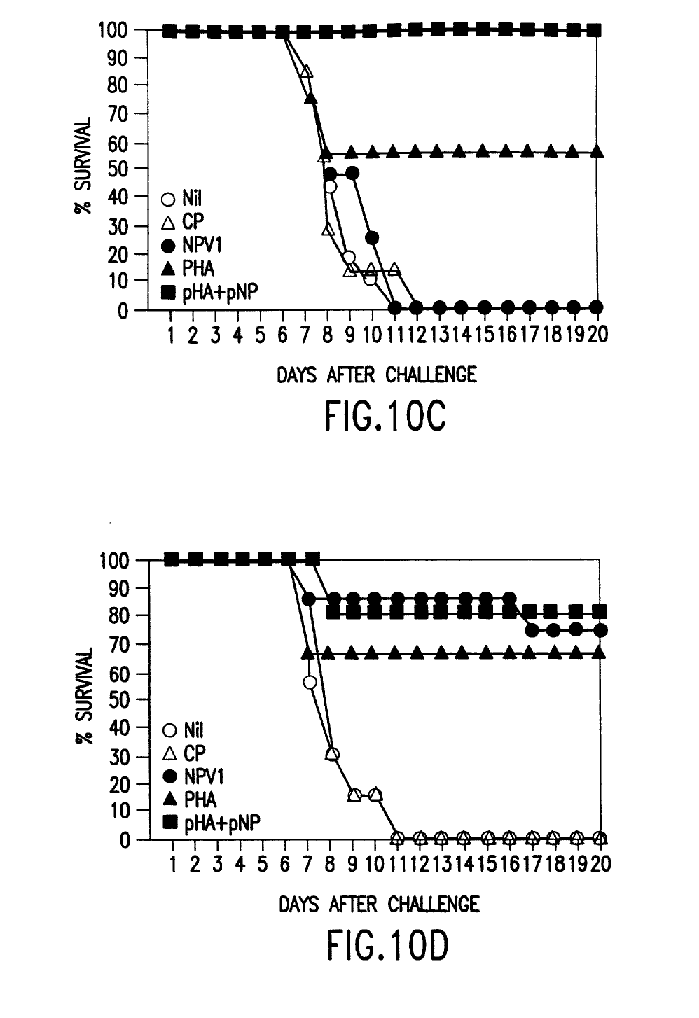

Enhanced protection against lethal challenge with Influenza virus by neonatal inoculation with pHA+pNP. FIG. 10 shows the protection against lethal challenge with WSN (A,C) or PR8 (B,D) virus of mice immunized as newborns (A,B) or adults (C,D) with a combination of pHA and pNP plasmids. As controls, we used naive mice, mice inoculated with a control plasmid (pRc/CMV) and mice immunized with pHA or pNP, separately. The mice were challenged with lethal doses of virus at four weeks following the completion of immunization. Newborn mice immunized with a dose of 25 μg+25 μg of pHA+pNP/inoculation and subsequently challenged with WSN virus displayed 100% survival, in spite of the fact that mice immunized only with pHA showed more than 50% mortality, or that mice immunized with pNP did not survive (FIG. 10A). Newborn and adult mice injected with control plasmid or non-immunized, displayed no survival when challenged with either WSN or PR8 virus, four weeks after the completion of immunization. Similarly, neonates immunized with the mixture of pHA and pNP displayed approximately 80% survival following lethal challenge with PR8 virus, compared to mice immunized with pHA or pNP alone, that showed approximately 25% and 15% survival, respectively (FIG. 10B). Adult mice immunized with both pHA and pNP were significantly more protected against WSN virus than adult mice immunized with either pHA or pNP alone (FIG. 10C). In contrast, mice immunized as adults with pNP+pHA displayed similar survival rates as compared to those immunized with pHA or pNP alone, following lethal challenge with PR8 virus (FIG. 10D) Together, these survival data show that coinjection of plasmids expressing HA and NP of Influenza virus type A into newborn mice greatly enhanced the protection against lethal infection with two distinct strains. This is more consistent with a synergistic rather than an additive relationship between HA and NP, due to the distinct nature of the immune effectors generated by the two components of the vaccine. These results were not only in contrast to the conventional view that newborn animals do not mount an immune response to vaccines, but also were surprising in that the synergistic effect was unexpected. The data indicate that combination vaccines according to the invention may be useful in creating a broader scope of protection to a pathogen, such as, for example, to encompass strain variations or genetic drift.

Dose dependency of protection following neonatal immunization with naked DNA. Further experiments were carried out in order to estimate the dose requirements for significant protection following neonatal immunization with plasmids expressing HA and NP of Influenza virus type A. Different groups of mice were inoculated with various doses of pHA, pNP or pHA+pNP. Control groups were inoculated with CP, representing the plasmid pRc/CMV lacking Influenza virus inserts. Four weeks after the completion of immunization, the mice were challenged with LD₁₀₀ of WSN virus. The number of mice that survived the challenge was recorded (Table 5) and the recovery of the surviving mice was demonstrated by the lack of pulmonary virus 16 days after the challenge. The mice were inoculated three times with plasmid. Administration of 25 μg of pHA together with 25 μg of pNP/dose resulted in complete protection, whereas inoculation of 50 μg of pHA or pNP was followed by approximately 50% and no protection, respectively (Table 5). In order to rule out the possibility of high zone tolerance in neonates, we immunized newborn mice with decreasing doses of pHA or pNP, separately. As shown in Table 5, the percentage of surviving mice decreased in the case of pHA and did not increase in the case of pNP. In contrast, adult or neonatal immunization with doses as small as 7 5 μg of each plasmid/dose was still followed by statistically significant protection after lethal challenge with WSN virus. Immunization of neonates with similar quantities of either pHA or pNP (15 μg/dose) induced no significant protection, further underlining the tremendous beneficial effect of associating the two plasmids in the same vaccine formulation.

TABLE 5
ENHANCED PROTECTION CONFERRED BY NEONATAL OR
ADULT IMMUNIZATION WITH A COMBINATION
OF HA AND NP EXPRESSING PLASMIDS

| Age of | | | | No. survivors/ | Percentage | |
|---|---|---|---|---|---|---|
| immuni- | Quantity (μg)/dose^{a} | | | total | survival | |
| zation | pHA | pNP | CP | infected | (%)^{b} | p value^{c} |
| Adult | — | — | — | 0/17 | 0 | — |
| | — | — | 50 | 0/7 | 0 | >0 1 |
| | 50 | — | — | 4/7 | 57 | 0 0003 |
| | — | 50 | — | 0/4 | 0 | >0.1 |
| | 25 | 25 | — | 5/5 | 100 | <0.0001 |
| | 15 | 15 | — | 6/6 | 100 | <0 0001 |
| | −5 | 7 5 | — | 6/7 | 86 | 0 0002 |
| Newborn | — | — | — | 0/10 | 0 | — |
| | — | — | 50 | 0/7 | 0 | >0 1 |
| | 50 | — | — | 5/12 | 42 | 0 01 |
| | 30 | — | — | 2/7 | 29 | >0 1 |
| | 15 | — | — | 1/6 | 17 | >0.1 |
| | — | 50 | — | 0/9 | 0 | >0 1 |
| | — | 15 | — | 0/4 | 0 | >0 1 |
| | 25 | 25 | — | 10 10 | 100 | <0 0001 |
| | 15 | 15 | | 5/6 | 83 | 0 0026 |
| | −5 | −5 | | 4/7 | 57 | 0.029 |
| | 3 | 3 | | 1/4 | 25 | >0 1 |

^{a}Mice were inoculated three times and challenged with WSN virus at 4 weeks after the completion of immunization
^{b}Survival was followed until day 20 after the challenge.
^{c}Statistical significance of survival as compared to the ml group was estimated by Fisher's exact test.

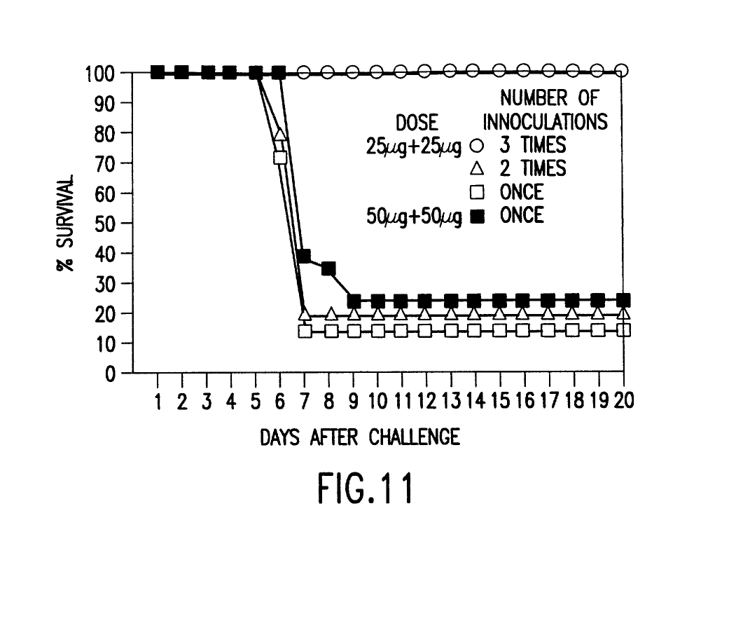

We studied the relationship between the number of inoculations and the protection conferred by neonatal immunization with pHA+pNP. Newborn mice were inoculated at day 1, 1 and 3, or 1, 3 and 6 with a mixture of pHA and pNP plasmids. At four weeks after the completion of immunization, the mice were challenged with a lethal dose of WSN virus. As shown in FIG. 11, one or two inoculations with 25 μg of each plasmid/dose, failed to induce significant protection. Even single inoculation of a larger dose of pHA together with pNP, did not result in significant protection. Thus, distribution of the naked DNA vaccine into multiple inoculations has beneficial effects in terms of protection.

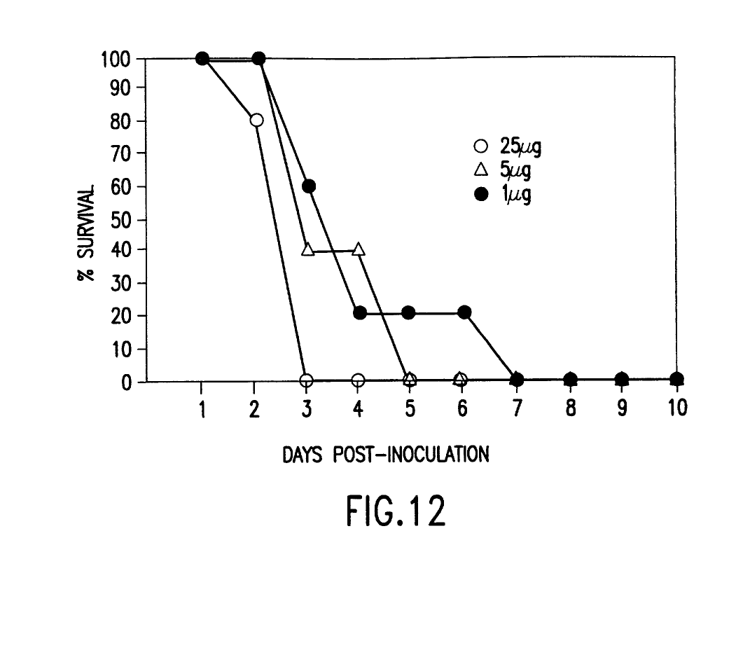

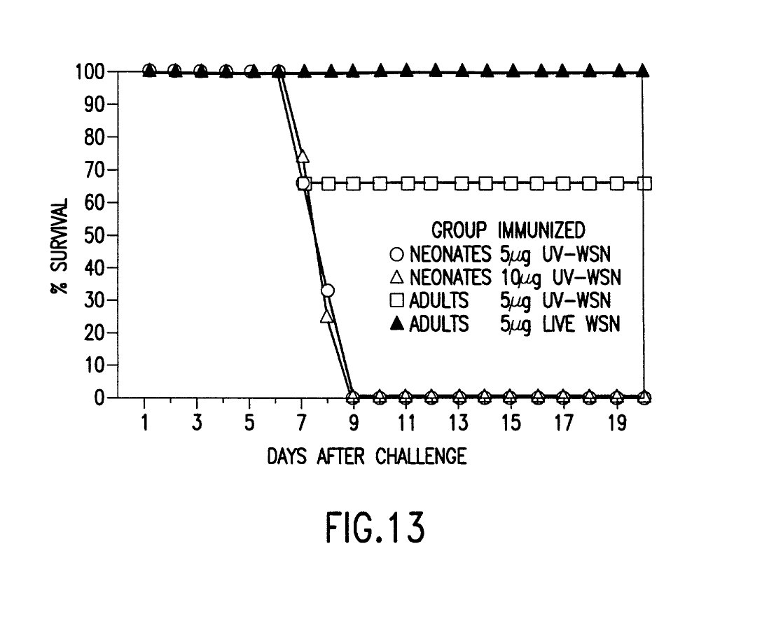

Lack of protection by neonatal immunization with UV-inactivated WSN virus. The observation described above, that live virus immunization of adult mice with WSN virus induced complete protection against homologous and heterologous challenge, correlated with the priming of a broad T and B cell response specific for the homologous strain as well as cross-reactive epitopes. We could not test the ability of WSN live-virus to induce protective immunity as when inoculated in newborn mice since the injection of this neurovirulent strain of Influenza virus into neonates was lethal at doses between less than 1 μg to 25 μg of sucrose purified virus. Invariably, the injection of WSN live virus in the gluteal region of 1day old BALB c mice was followed by impaired thriving beginning with 24-48 hours after inoculation and culminating with dehydration and death at 3 to 5 days postinjcction (FIG. 12) Distinct batches of WSN virus displayed less pronounced but significant and reproducible lethality in terms of percentage survivors. Consequently, we carried out further experiments with UV-attenuated WSN virus, that is similar to the conventional killed Influenza virus vaccine. In sharp contrast to the adult mice immunized with UV-attenuated WSN virus, the neonates although surviving the immunization, were not protected against the challenge (four weeks later) with LD₁₀₀ of WSN virus (FIG. 13) This is consistent with the lack of CTL response, the deviated Th response and the B cell tolerance following neonatal inoculation of UV attenuated WSN virus, as shown above

Clearance of the pulmonary virus in mice immunized as newborns with pHA+pNP. Immunization of adult mice with live WSN virus leads to generation of optimal titers of protective antibodies specific for the homologous strain (Table 4). A subsequent exposure to the same strain of virus does not lead to infection due to the presence of hemagglutination inhibiting antibodies, that prevent the virus binding to the sialoreceptors on the epithelial cells of the respiratory tract. Indeed, no pulmonary virus could be detected as early as three days after homologous challenge of mice immunized with live WSN virus (Table 6). In contrast, non-immunized mice or mice injected with CP as adults or neonates displayed significant pulmonary virus titers at day 3 and 7 after infection. All of the mice immunized with pHA+pNP as adults or newborns, although displaying significant pulmonary virus at day 3, showed no virus at day 7 following infection with WSN virus (Table 6). Furthermore, the mice immunized with pHA successfully cleared the virus by day 7. However, not all the mice immunized with pHA survived the challenge (FIG. 10), probably because of the extensive DTH reaction due to delayed clearance of the virus. Together, these data suggest that, while the plasmid immunization did not induce optimal titers of neutralizing antibodies capable to prevent the homologous infection, the T cell memory response led to effective clearance of the virus, in mice immunized either as adults or as neonates with pHA+pNP.

TABLE 6
CLEARANCE OF THE PULMONARY VIRUS BY MICE
IMMUNIZED AS NEONATES OR ADULTS WITH A
COMBINATION OF PLASMIDS EXPRESSING HA AND NP

| | |
|---|---|
| Age of | Log₁₀ of TCID₅₀ (mean ± SE)^{a} |

| immunization | day 3 | day 7 | day 20 |
|---|---|---|---|
| Adult mice | | | |
| injected with | | | |
| Nil | 5 4 ± 0 | 3 7 ± 0 3 | +^{b} |
| CP | 4 9 ± 0 5 | 2.8 ± 0 5 | + |
| WSN virus | <1 0 | <1.0 | <1 0 |
| NPV1 | 4 8 ± 1 | + | |
| pHA | 2 0 ± 2 2 | 1 4 ± 0 8 | <1.0 |
| NPV1-pHA | 4 4 ± 1 1 | <1.0 | <1 0 |
| Newborn mice | | | |
| injected with | | | |
| CP | 4 2 ± 0 5 | + | + |
| NPV1 | 4 | + | + |
| pHA | 4 0 ± 0 0 | <1 0 | <1.0 |
| NPV1-pHA | 3 4 ± 1 2 | <1 0 | <1 0 |

^{a}At day 3 and 7 after the lethal challenge with WSN virus, the pulmonary virus titers were estimated.
At day 20, all the surviving mice were immunized and the lung titers measured
^{b}No surviving mice
^{c}Titers were considered lower than Nil infectious virus was not detected

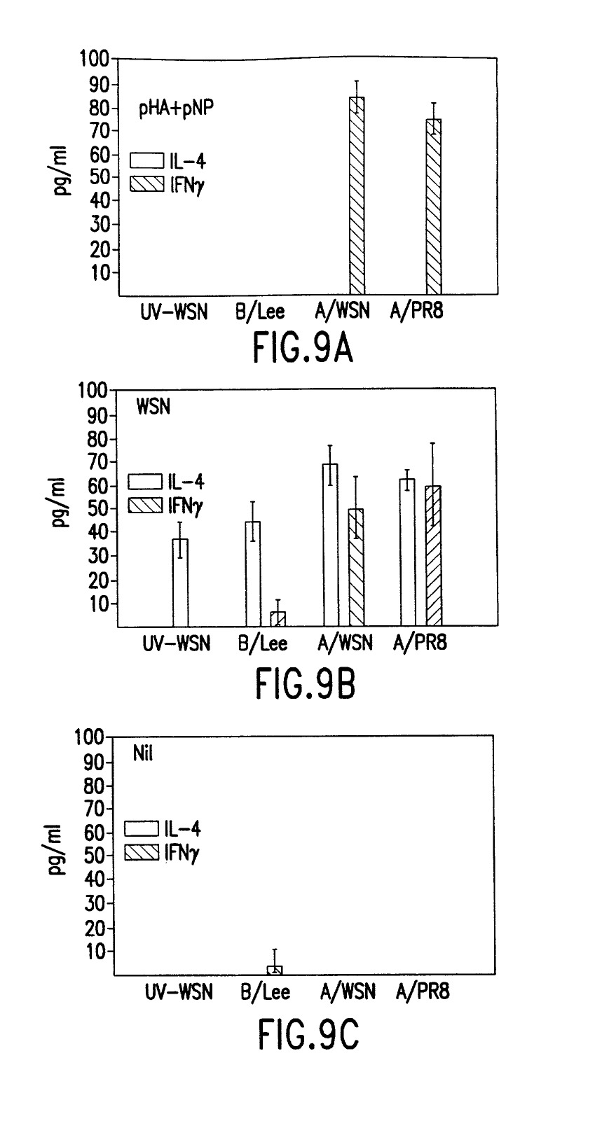

Conclusion. In contrast to neonatal inoculation of UV-attenuated WSN virus, which is similar to the conventional killed vaccine (that fails to trigger a protective immune response), we show that neonatal coadministration of two plasmids expressing NP (pNP) and HA (pHA) induces protection against lethal challenge with the homologous virus and a drift variant.

Whereas HA bears dominant B and Th epitopes that are mostly strain or subtype specific, NP carries major cross-reactive CTL epitopes. Neonatal inoculation of pHA+pNP was followed by induction of CTLs that displayed cross-reactivity against various type A strains (FIG. 8). Furthermore, neonatal DNA immunization induced CD4⁺Th1 cells specific for epitopes shared by WSN virus and the drift variant, PR8 virus (FIG. 9). Finally, DNA immunization of newborn mice elicited protective antibodies against the homologous strain of virus, that was used for cloning the HA insert from pHA (Table 4). However, only 5 out of 8 mice were responders and the HI titers were significantly reduced as compared to adult mice immunized with live or UV-attenuated virus. Together, the virus-specific CTL, Th and B cells mediated a significantly increased protection against lethal challenge with WSN, in mice immunized as neonates or as adults (FIG. 10). In the case of the drift variant PR8 virus, the enhanced protection was due to the induction of PR8 specific Th and CTL, since no PR8 specific HI antibodies were measured (Table 4). The dose-protection relationship shown in Table 5, suggests strong synergism between the main immune effectors since lower doses of pHA+pNP were sufficient to induce levels of protection that could not be obtained with either pHA or pNP In particular, although pNP elicited CTL against the major epitope NP 147-155 shared by PR8 and WSN virus, it failed to induce significant protection in terms of survival against the lethal challenge with WSN virus. The most reasonable explanation is the enhanced virulence associated with increased replication of the WSN strain due to a mutation in neuraminidase, so that CTL alone are not sufficient for significant protection against this particular strain.

In stark contrast with neonatal immunization with pHA+pNP, inoculation of WSN virus was not followed by protection. First, injection of live WSN virus in newborn mice was lethal (FIG. 12) Since inoculation of live WSN virus in adult mice was not lethal and induced complete protection against homologous challenge, this result supports the concern that live viral vaccines may induce serious side effects due to the immaturity of the neonatal immune system. Secondly, neonatal inoculation with UV-attenuated WSN virus, although not lethal because of the impairment of virus replication, did not elicit protection (FIG. 13). No CTL or B cells secreting protective antibodies were primed by UV-attenuated virus inoculated into newborn mice (FIG. 8 and Table 4). Whereas the lack of cytotoxicity may be easily explained by the lack of synthesis of viral proteins, the absence of an humoral response is most probably due to the immaturity of the neonatal immune system since adults mounted HI antibodies to UV-attenuated WSN virus. Indeed, recent data suggest that neonatal exposure to UV-attenuated WSN virus induces B cell tolerance. Further, neonatal inoculation with UV-attenuated virus induced CD4⁺Th cells that secreted IFNγ and IL-4 (FIG. 10). It is not clear at this point how much of the IL-4 is due to the Th cells specific for culture media proteins, although we used for immunization virus purified by sucrose-gradient ultracentrifugation. It is noteworthy to mention that immunization of adult mice with UV-attenuated WSN virus, in contrast to neonatal immunization, resulted in significant but not complete protection to homologous challenge (FIG. 13). Thus, neonatal and adult immunization with UV-attenuated Influenza virus, that is similar to the conventional vaccine, appears to be less effective as compared to DNA immunization with mixtures of plasmids encoding multiple Influenza antigens.

In conclusion, neonatal inoculation of plasmids expressing HA and NP of Influenza virus was followed by priming of CTL, Th and B cells as well as increased protection against lethal challenge with two strains of virus. The data indicate that, rather than having a tolerizing effect. T cell immunity and humoral immunity are induced by neonatal DNA inoculation In contrast, neonatal immunization with UV-attenuated WSN virus (analogous to a conventional vaccine) did not induce protection and live-virus inoculation of newborn mice was lethal

### 8. EXAMPLE

### Immune Responsiveness following Neonatal Inoculation with a Plasmid Expressing an Ig Chimera Bearing T and B Epitopes of Hemagglutinin

It has been shown that self immunoglobulin molecules are effective vehicles for delivering foreign epitopes to MHC class-II molecules in the endosomal compartment of professional APC. We have engineered a chimeric gene by replacing the CDR3 and CDR2 segments of the VH fragment from an anti-arsonate mAb with the gene segments encoding major HA epitopes: HA 110-120 that is recognized by CD4⁺T cells in the context of I-E^{d} class-II molecules and HA 150-159 respectively, that is a B cell epitope. Subsequently, the VH-TB chimeric gene was inserted into a mammalian expression vector bearing the CMV initial-early promoter and the BGH poly-adenylation signal. Further studies showed that myoblast cells transfected with the VH-TB plasmid secrete the chimeric protein in the supernatant.

Recent studies showed that neonatal inoculation with plasmids expressing the circumsporozoite antigen of Plasmodium yoelli induced tolerance to major epitopes previously defined in adults but not to non-dominant epitopes (Mor et al. 1996, J. Clin. Invest. 98:2700). We used the VH-TB chimera that bears defined T and B cell Influenza virus epitopes to inoculate neonatal mice and tested the priming effect of the VH-TB plasmid subsequent to the inoculation in adult or newborn mice.

The immune response generated by adult immunization with VH-TB plasmid. Adult BALB c mice immunized with VH-TB plasmid develop both T and B cell immunity (Table 7) The CD4 T cells separated from adult mice immunized with VH-TB at day 7 after the completion of immunization, secreted significant amounts of IFNγ but no IL-4 when restimulated with PR8 virus or a construct bearing the HA 110-120 peptide. In contrast, CD4 T cells from PR8 immunized mice secreted both IFNγ and IL-4. Adult mice immunized with VH-TB mounted HA 150-159 specific antibodies at 4 weeks following the completion of immunization (Table 7). The titers of HA 150-159 and PR8-specific antibodies measured in VH-TB immunized adult mice were significantly lower than those of the mice immunized with live PR8 virus. Thus, VH-TB immunization of adult mice induced immune responses to the Th as well as the B cell epitope encoded by the chimeric gene.

TABLE 7
THE IMMUNE RESPONSE OF ADULT MICE TO VH-TB PLASMID

| | Cytokine production by CD4 T cells | | | | | | | | | |
|---|---|---|---|---|---|---|---|---|---|---|
| | In vitro stimulation with^{a} | | | | | | | | Antibody response^{b} | |
| Mice immunized | Nil | | lgG2b | | lgG-gal-HA | | PR8 virus | | anti- | anti-HA |
| with | IFNγ | IL-4 | IFNγ | IL-4 | IFNγ | IL-4 | IFNγ | IL-4 | PR8 | 150-159 |
| Nil | 0 | 0 | 0 | 0 | 0 | 0 | 0 | 0 | 0 | 0 |
| B/Lee/40 virus | 0 | 0 | 0 | 0 | 0 | 0 | 0 | 0 | 0 | 0 |
| PR/8/34 virus | 0 | 0 | ND | ND | ND | ND | 74 ± 3 | 24 ± 3 | 42 ± 9 | 12 ± 4 |
| VH-TB | 0 | 0 | 0 | 0 | 39 ± 6 | 0 | 56 ± 3 | 0 | 4 ± 2 | 5 ± 3 |
| plasmid | | | | | | | | | | |

^{a}Negatively selected CD4 T cells were restimulated for four days in the presence of 5 μg/ml of antigen The concentration of cytokines in the supernatant was determined by ELISA and expressed as mean ± SD of duplicates in pg/ml.
^{b}The binding of antibodies to PR8 or HA 150-159 coupled to BSA was estimated by sandwich RIA using 1 100 dilutions of sera and iodinated rat anti-mouse k light chain antibodies The standard curve was constructed using B2H1 HA-specific antibodies Results were expressed as mean ± SD of triplicates (μg/ml)
^{c}ND—not done

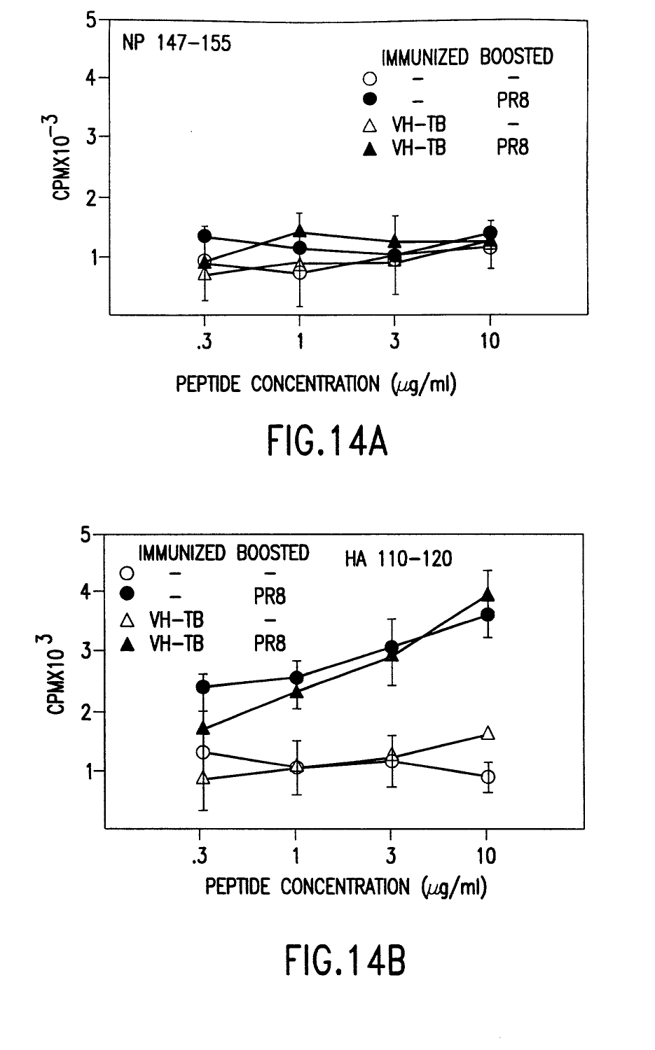

Cellular responsiveness subsequent to the neonatal inoculation of VH-TB plasmid. We separated CD4⁺ T cells from 4 week-old mice immunized as neonates with VH-TB and we tested their proliferation upon in vitro stimulation with HA 110-120 peptide or NP 147-155 peptide. Negatively selected CD4+ cells from mice immunized with VH-TB as neonates were incubated with APC in the presence of various concentrations of Np 147-155 or HA 110-120 synthetic peptides. Tritiated thymidine was added after 72 hours and the radioactivity incorporated was measured after another 14 hours. The results are expressed as means of triplicates, plus or minus the standard deviation of proliferation idices. Some mice immunized with VH-TB were boosted with PR8 virus. As controls, we used naive age-matched mice and mice immunized with live PR8 virus one week prior to sacrifice. Some of the mice were boosted with live PR8 virus at the age of 3 weeks, in order to address the question of tolerance induction. As shown in FIG. 14B, the CD4⁺ T cells from mice immunized as neonates with VH-TB and boosted with PR8 virus proliferated to a similar extent as the CD4⁺ T cells from mice immunized with live-virus at the age of 3 weeks. In contrast, the CD4⁺ T cells from non-immunized mice or mice immunized as newborns with VH-TB did not proliferate when restimulated with HA 110-120 peptide. No significant proliferation was measured when the CD4⁺ T cells where stimulated with NP 147-155 peptide, that is a major H-2 K^{d} epitope (FIG. 14A).

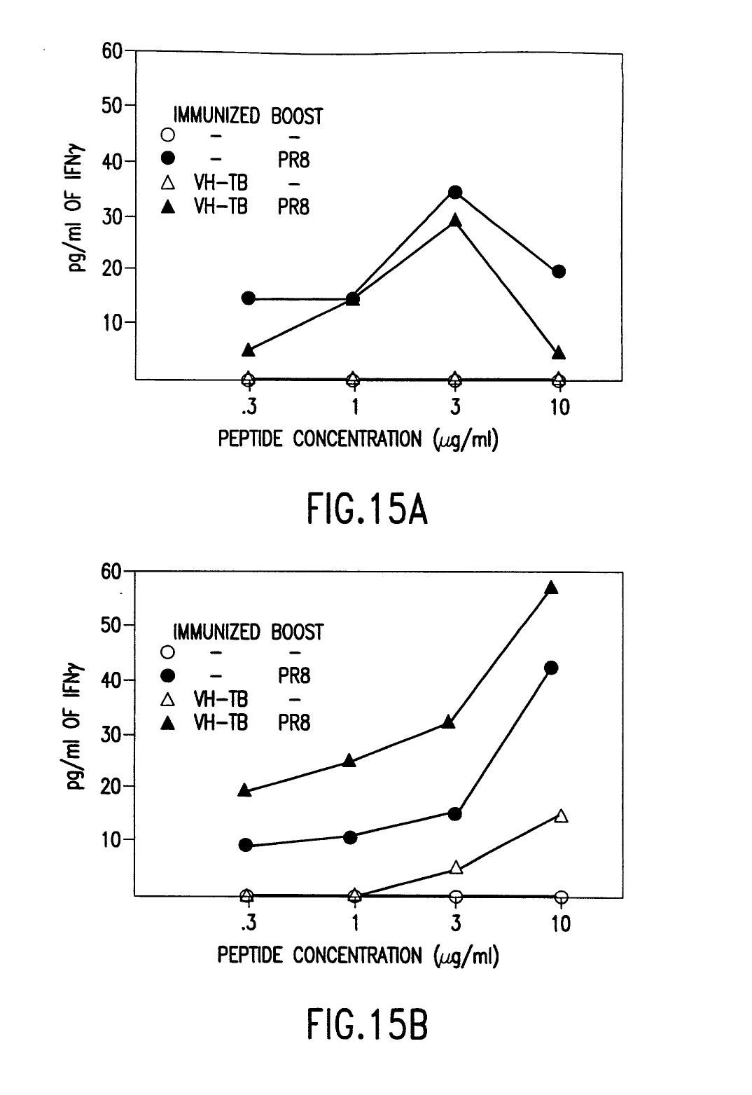

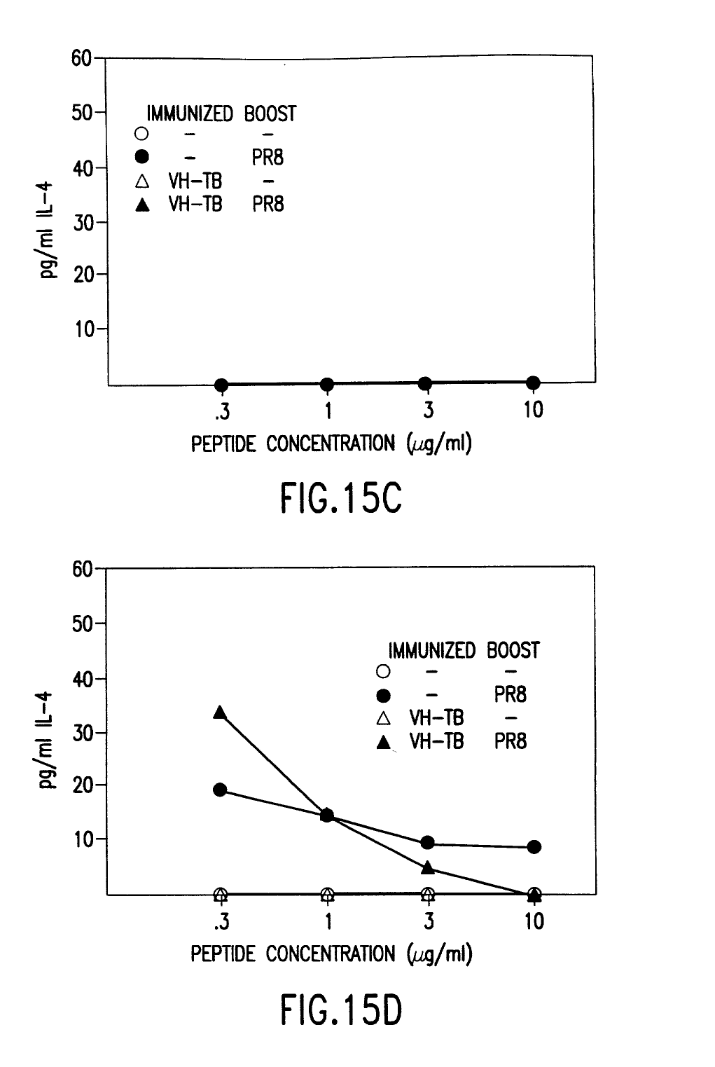

We tested the ability of nylon-wool purified T cells to produce cytokines following in vitro stimulation as with NP 147-155 or HA 110-120 peptide. Specifically, nylon wool purified T cells from spleens of mice immunized as neonates with VH-TB were incubated with various concentrations of NP 147-155 (A.C) or HA 110-120 (B.D) synthetic peptides in the presence of APC and 6 U/ml rIL-2. IFNγ (A.B) and IL-4 (C<D) were measured three days later by ELISA and the results were expressed as means of duplicates (pg/ml). SE was less than 25% of the mean in each case. As controls, we used naive mice and mice immunized with PR8 virus one week prior to sacrifice. Part of the mice immunized with VH-TB were boosted with PR8 virus one week before the study. The T cells from mice that received an inoculation with live PR8 virus, previously immunized or not with VH-TB, secreted significant IFNγ but no IL-4 when restimulated with NP 147-155 peptide (FIGS. 15A,C). Furthermore, significant amounts of IFNγ and IL-4 were produced by T cells from mice injected with live-virus, that were previously immunized or not with VH-TB as neonates (FIGS. 15 B,D). The T cells from mice immunized as neonates with VH-TB and not boosted with PR8 virus secreted low but measurable amounts of IFNγ when in vitro stimulated with HA 110-120 peptide (FIG. 15B). Interestingly, the T cells from mice immunized with live-virus displayed dissimilar profiles of IFNγ and IL-4 secretion depending on the concentration of HA 110-120 peptide: whereas at lower concentrations IL-4 dominated, at higher concentrations the T cells produced more IFNγ and less IL-4.

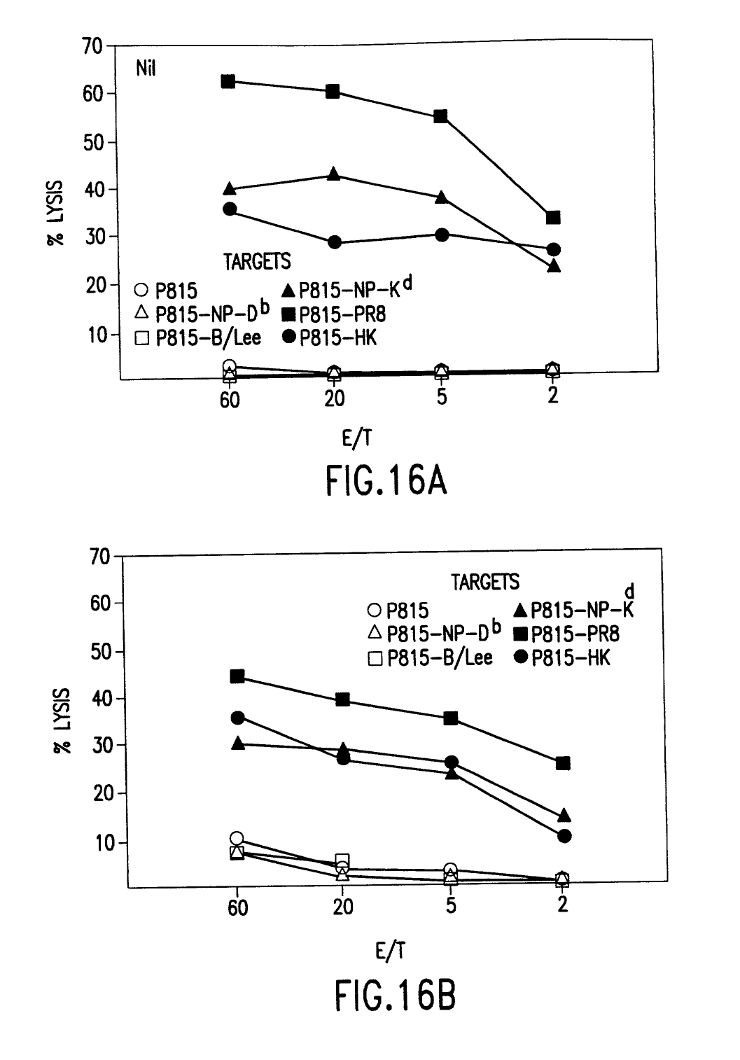

In further experiments, mice were immunized with VH-TB as newborns and boosted three weeks later with live PR8 virus. The splenocytes from three mice in each group were harvested and pooled (see FIG. 16) one week later and in vitro stimulated with various strains of influenza or coated with NP synthetic peptides. The results are expressed as means of percent specific lysis of duplicates. The mice inoculated as neonates with VH-TB mounted significant cytotoxicity subsequent to live PR8 virus boost (FIG. 16). Splenocytes harvested from mice injected with live virus and previously immunized or not with VH-TB, after in vitro stimulation with PR8 virus, lysed the target cells infected with PR8 or HK virus, or coated with NP 147-155 peptide. Thus, neonatal inoculation with VH-TB did not impair a subsequent T cell response to the live PR8 virus.

Humoral responsiveness following the neonatal inoculation of VH-TB plasmid. Neonatal inoculation of the VH-TB plasmid was not followed by the induction of humoral responses, as revealed by the lack of PR8 neutralizing antibodies (Table 8). The binding activity for HA 150-159 peptide or PR8 virus of the sera of mice immunized as newborns with VH-TB, was similar to that of naive mice (Table 8). Neonatal injection of VH-TB plasmid did not induce unresponsiveness to PR8 virus, since mice boosted with live-virus showed unaffected neutralizing responses. Furthermore, the response to the HA 150-159 peptide, that is a major B cell epitope expressed by VH-TB, was not impaired by neonatal inoculation of the plasmid, as revealed by the ELISA data (Table 8)

TABLE 12
THE HUMORAL RESPONSIVENESS OF MICE
IMMUNIZED AS NEONATES WITH VH-TB PLASMID

| Group | | HI titer against | Binding to^{b}: | |
|---|---|---|---|---|
| Immunized | Boost | PR8 virus^{a} | HA 150-159 | PR8 virus |
| — | — | 0 | 177 ± 33 | 163 ± 20 |
| VH-TB | — | 0 | 175 ± 61 | 183 ± 17 |
| — | PR8 | 7 0 ± 1 0 | 352 ± 48 | 337 ± 79 |
| VH-TB | PR8 | 6 0 ± 0 7 | 308 ± 39 | 354 ± 26 |

^{a}Results were expressed as mean ± SE of log₂ HI titers. HI titers less than 40 were considered 0
^{b}The binding of antibodies to the B epitope and PR8 virus was estimated by sandwich ELISA using sera at a dilution of 1/200 and biotin-conjugated goat anti-mouse IgG antibody Results were expressed as mean ± SEM of OD₄₅₀.

Conclusion. The foregoing studies show that mice injected as newborns with VH-TB and boosted with PR8 virus developed:

1) antibodies that are neutralizing for PR8 virus and bind the HA 150-159 peptide (Table 8);

2) T cells that secreted IFNγ and IL-4 following in vitro stimulation with HA 110-120 peptide (FIG. 15);

3) CD4⁺ T cells that proliferated upon in vitro stimulation with HA 110-120 peptide (FIG. 14);

4) CTLs that lysed target cells infected with type A Influenza viruses or coated with NP 147-155 peptide that is not carried by VH-TB (FIG. 10).

These results demonstrate that neonatal inoculation of VH-TB may enhance Th or CTL responses in the case of subsequent exposure to live virus. Although neonates responded less effectively than the adults to VH-TB, the fact that they mounted an immune response remains surprising in view of conventional notions of the tolerizing, rather than immunizing, effect of neonatal vaccination. Accordingly, this example demonstrates that the DNA vaccine compositions of the invention may be used with conventional vaccination procedures to provide an enhanced immune response comprising both T-cell and humoral components.

Various publications are cited herein, the contents of which are incorporated by reference in their entireties.

1. A method for immunizing an infant mammal against a target antigen, comprising inoculating the mammal with an effective amount of a nucleic acid encoding a relevant epitope of the target antigen in a pharmaceutically acceptable carrier, such that a therapeutically effective amount of the relevant epitope is expressed in the infant mammal.

2. The method of claim 1, wherein the target antigen is a viral antigen.

3. The method of claim 1, wherein the target antigen is a bacterial antigen.

4. The method of claim 2, wherein the target antigen is a respiratory syncytial virus antigen.

5. The method of claim 2, wherein the target antigen is a rotavirus antigen.

6. The method of claim 2, herein the target antigen is a measles virus antigen.

7. The method of claim 2, wherein the target antigen is a human immunodeficiency virus antigen.

8. The method of claim 2, wherein the target antigen is a hepatitis virus antigen.

9. The method of claim 2, wherein the target antigen is a hepatitis B virus antigen.

10. The method of claim 2, wherein the target antigen is a herpes simplex virus antigen.

11. The method of claim 2, wherein the target antigen is an influenza virus antigen.

12. The method of claim 3, wherein the target antigen is a Streptococcus pneumoniae antigen.

13. The method of claim 3, wherein the target antigen is a Hemophilus influenzae antigen.

14. The method of claim 3, wherein the target antigen is a Neisseria meningitidis antigen.

15. The method of claim 3, wherein the target antigen is a Staphylococcus aureus antigen.

16. The method of claim 1, wherein the target antigen is a protozoan antigen.

17. The method of claim 16, wherein the target antigen is a malaria antigen.

18. A method for inducing a cytotoxic T cell response to a target antigen in an infant mammal, comprising inoculating the mammal with an effective amount of a nucleic acid encoding a relevant epitope of the target antigen in a pharmaceutically acceptable carrier, such that a therapeutically effective amount of the relevant epitope is expressed in the infant mammal.

19. The method of claim 19, wherein the infant mammal carries a detectable amount of maternal antibodies

20. A method for immunizing an infant mammal against a pathogen comprising inoculating the mammal with an effective amount of nucleic acid encoding more than one relevant epitope of one or more target antigen associated with the pathogen in a pharmaceutically acceptable carrier, such that therapeutically effective amounts of the relevant epitopes are expressed in the infant mammal.

21. The method of claim 20, wherein the relevant epitopes are encoded by the same nucleic acid molecule.

22. The method of claim 20, wherein the relevant epitopes are encoded by different nucleic acid molecules.

23. The method of claim 20, wherein the pathogen is an influenza virus.

24. A method for inducing a cytotoxic T cell response against a pathogen in an infant mammal, comprising inoculating the mammal with an effective amount of nucleic acid encoding more than one relevant epitope of one or more target antigen associated with the pathogen in a pharmaceutically acceptable carrier, such that therapeutically effective amounts of the relevant epitopes are expressed in the infant mammal

25. The method of claim 24, wherein the target antigen is a viral antigen.

26. The method of claim 24, wherein the target antigen is a bacterial antigen.

27. The method of claim 25, wherein the target antigen is a respiratory, syncytial virus antigen.

28. The method of claim 25, wherein the target antigen is a rotavirus antigen.

29. The method of claim 25, wherein the target antigen is a measles virus antigen.

30. The method of claim 25, wherein the target antigen is a human immunodeficiency virus antigen.

31. The method of claim 25, wherein the target antigen is a hepatitis virus antigen.

32. The method of claim 31, wherein the target antigen is a hepatitis B virus antigen.

33. The method of claim 25, wherein the target antigen is a herpes simplex virus antigen.

34. The method of claim 25, wherein the target antigen is an influenza virus antigen.

35. The method of claim 26, wherein the target antigen is a Streptococcus pneumoniae antigen.

36. The method of claim 26, wherein the target antigen is a Hemophilus influenzae antigen.

37. The method of claim 26, wherein the target antigen is a Neisseria meningitidis antigen.

38. The method of claim 26, wherein the target antigen is a Staphlyococcus aureus antigen.

39. The method of claim 24, wherein the target antigen is a protozoan antigen.

40. The method of claim 39, wherein the target antigen is a malaria antigen.

41. A composition of nucleic acid encoding one or more relevant epitopes of one or more target antigens, for use in the preparation of a vaccine for use in an infant mammal.

42. A composition of nucleic acid encoding one or more relevant epitopes of one or more target antigens, for use in the preparation of an immunogenic composition which may be used in a method of inducing a cellular immune response in an infant mammal.

43. A method for immunizing an infant mammal having an immune response which has susceptibility to high-zone tolerance against a target antigen, comprising inoculating the mammal with an effective amount of a nucleic acid encoding a relevant epitope of the target antigen in a pharmaceutically acceptable carrier, such that a therapeutically effective amount of the relevant epitope is expressed in the infant mammal.

44. A method for immunizing an infant mammal having an immune response which has a humoral response of reduced magnitude and restricted isotype, comprising inoculating the mammal with an effective amount of a nucleic acid encoding a relevant epitope of the target antigen in a pharmaceutically acceptable carrier, such that a therapeutically effective amount of the relevant epitope is expressed in the infant mammal.

45. A method for immunizing an infant mammal having an immune response which has a Th2 biased helper response, comprising inoculating the mammal with an effective amount of a nucleic acid encoding a relevant epitope of the target antigen in a pharmaceutically acceptable carrier, such that a therapeutically effective amount of the relevant epitope is expressed in the infant mammal.

46. A method for immunizing an infant mammal having an immune response which has a cellular immune response of reduced magnitude, comprising inoculating the mammal with an effective amount of a nucleic acid encoding a relevant epitope of the target antigen in a pharmaceutically acceptable carrier, such that a therapeutically effective amount of the relevant epitope is expressed in the infant mammal.

47. A method of increasing the level of maternal antibodies to a target antigen in an infant mammal, comprising immunizing the pregnant mother with an effective amount of a nucleic acid encoding a relevant epitope of the target antigen in a pharmaceutically acceptable carrier, such that a therapeutically effective amount of the relevant epitope is expressed in the infant mammal.


SEQUENCE LISTING (20 sequences)

SEQ ID NO: 1 - protein, 19 aa, Human Immunodefficiency Virus Type 1
  301...319: Envelope Protein gp120
RKSIHIGPGRAFYTTGEII

SEQ ID NO: 2 - protein, 10 aa, Influenza Virus
  HA1 hemagglutinin protein
WLTKKGDSYP

SEQ ID NO: 3 - protein, 10 aa, Influenza Virus
  H3 protein
WLTKSGSTYP

SEQ ID NO: 4 - protein, 10 aa, Influenza Virus
  H2 protein
WLTKEGSDYP

SEQ ID NO: 5 - protein, 11 aa, Measles Virus
  404...414: F protein
INQDPDKILTY

SEQ ID NO: 6 - protein, 19 aa, Foot and Mouth Disease Virus
  141...160: VP1 protein
MNSAPNLRGDLQKVARTLP

SEQ ID NO: 7 - protein, 11 aa, Influenza PR8A Virus
  110...120: Hemagglutinin Protein
SFERFEIFPKE

SEQ ID NO: 8 - protein, 20 aa, <Unknown>
  Tetanus Toxoid Protein
NSVDDALINSTKIYSYFPSV

SEQ ID NO: 9 - protein, 17 aa, <Unknown>
  Tetanus Toxoid
PEINGKAIHLVNNESSE

SEQ ID NO: 10 - protein, 15 aa, <Unknown>
  88...103: Cytochrome C Protein
ANERADLIAYLQATK

SEQ ID NO: 11 - protein, 20 aa, Mycobacteria
  350...369: Heat Shock Protein
DQVHFQPLPPAVVKLSDALI

SEQ ID NO: 12 - protein, 14 aa, Hen
  48...61: Egg White Lysozyme
DGSTDYGILQINSR

SEQ ID NO: 13 - protein, 12 aa, Streptococcus A
  308...319: M Protein
QVEKALEEANSK

SEQ ID NO: 14 - protein, 20 aa, Staphylococcus sp.
  81...100: Nuclease Protein
RTDKYGRGLAYIYADGKMVN

SEQ ID NO: 15 - protein, 15 aa, Influenza PR8A Virus
  147...161: NP Protein
TYQRTRALVRTGMDP

SEQ ID NO: 16 - protein, 15 aa, Influenza Virus
  365-379: NP protein
IASNENMDAMESSTL

SEQ ID NO: 17 - protein, 9 aa, <Unknown>
  33-41: LSMV peptide
KAVYNFATM

SEQ ID NO: 18 - protein, 8 aa, <Unknown>
  257-264: ovalbumin
SIINFEKL

SEQ ID NO: 19 - DNA (cDNA), 30 bp, <Unknown>; single-stranded, linear
  primer
cattgtctagaatttgaactcctctagtgg

SEQ ID NO: 20 - DNA (cDNA), 17 bp, <Unknown>; single-stranded, linear
  primer
aatttgaatgatgcaac